(19) United States
(12) Patent Application Publication
(10) Pub. No.: US 2017/0335314 A1
(43) Pub. Date: Nov. 23, 2017

(54) Methods For Using Nucleic Acid Aptamers For Directed Templated Assembly

(71) Applicant: TriBiotica LLC, Madison, WI (US)

(72) Inventors: Ian Dunn, Beverly, MA (US); Matthew Lawler, Madison, WI (US)

(21) Appl. No.: 15/601,449

(22) Filed: May 22, 2017

Related U.S. Application Data
(60) Provisional application No. 62/339,981, filed on May 23, 2016.

(51) Int. Cl. C12N 15/10; A61K 31/713; C12N 15/115

(52) U.S. Cl. CPC C12N 15/1048; C12N 15/115; A61K 31/713; C12N 2310/531; C12N 2310/16

(57) ABSTRACT

The present disclosure provides nucleic acid aptamers, nucleic acid aptamers hybridized to haplomers, methods of using nucleic acid aptamers to present template sequences, where the aptamers bind to target molecules unique to specific cellular targets, for the purpose of nucleic acid-templated assembly of molecules with desired functions.

### CROSS-REFERENCE TO RELATED APPLICATIONS

This application claims priority to U.S. provisional application Ser. No. 62/339,981 filed May 23, 2016, which is incorporated herein by reference in its entirety.

### FIELD

The present disclosure is directed, in part, to methods of using nucleic acid aptamers to present template sequences, where the aptamers bind to target molecules unique to specific cellular targets, for the purpose of nucleic acid-templated assembly of molecules with desired functions.

### BACKGROUND

A goal of drug development is delivering potent bio-therapeutic interventions to pathogenic cells, such as virus infected cells, neoplastic cells, cells producing an autoimmune response, and other dysregulated or dysfunctional cells. Examples of potent bio-therapeutic interventions capable of combating pathogenic cells include toxins, pro-apoptotic agents, and immunotherapy approaches that re-direct immune cells to eliminate pathogenic cells. Unfortunately, developing these agents is extremely difficult because of the high risk of toxicity to adjacent normal cells or the overall health of the patient.

A method that has emerged to allow delivery of potent interventions to pathogenic cells while mitigating toxicity to normal cells is targeting of therapeutics by directing them against molecular markers specific for pathogenic cells. Targeted therapeutics have shown extraordinary clinical results in restricted cases, but are currently limited in their applicability due to a lack of accessible markers for targeted therapy. It is extremely difficult, and often impossible, to discover protein markers for many pathogenic cell types.

More recently, therapies targeted to nucleic acid targets specific to pathogenic cells have been developed. Existing nucleic acid-targeted therapies, such as siRNA, are able to down-modulate expression of potentially dangerous genes, but do not deliver potent cytotoxic or cytostatic interventions and thus are not particularly efficient at eliminating the dangerous cells themselves. Hence, there exists a need to combat the poor efficacy and/or severe side effects of existing bio-therapeutic interventions.

Finding proximal binding sites in proteins or other macromolecules cannot be performed according to simple hybridization rules. Rather than a readily applied digital code, the ligand binding can be seen as an analog process, where the ligand and its receptor pocket share a shape-based complementarity. Rational design of such ligand-mediated templating therefore requires detailed three-dimensional structural information. Even where crystal structures of proteins (considered as possible target templates) is available, design of interactive ligands is another step upward in difficulty, especially where such ligands must bind within tightly proscribed spatial boundaries relative to each other. Moreover, such design must also take into account the possibility of binding-related conformational changes (akin to allostery), which could inadvertently destroy the desired spatial proximity. While these caveats do not rule out testing specific protein choices for templating purposes, they do emphasize the difficulties of finding non-nucleic acid templates in target aberrant cells in realistic time-frames.

Although much progress has been made in recent years with respect to therapy for specific cancers, a great many therapeutic gaps still exist. Such unmet needs for better treatments are highly applicable to many tumor types. Moreover, a general therapy capable of targeting specific pathological or undesirable cells is desired to be extended into much broader therapeutic domains, including allergy and autoimmunity.

### Summary

In general, the present disclosure provides methods for generating aptamers against target molecules of a variety of classes, where the aptamers provide templating sequences to adapt the target molecules for effector partial/haplomers, for the assembly of peptides or other structures for diagnostic or therapeutic applications.

In particular, the present disclosure provides singlet nucleic acid aptamers comprising a first portion folded into a tertiary structure that is able to bind to a target molecule, and a second portion comprising either the 3′ or 5′ terminal region, wherein the second portion is hybridized to a first haplomer and a second haplomer. The first haplomer comprises a hybridization region that is hybridized to the second portion of the singlet nucleic acid aptamer, and a reactive effector moiety. The second haplomer comprises a hybridization region that is hybridized to the second portion of the singlet nucleic acid aptamer, and a reactive effector moiety. The reactive effector moiety of the first haplomer is in spatial proximity to the reactive effector moiety of the second haplomer.

The present disclosure also provides dual proximal nucleic acid aptamer pairs comprising a first nucleic acid aptamer and a second nucleic acid aptamer. The first nucleic acid aptamer comprises a first portion folded into a tertiary structure that is able to bind to a target molecule, and a second portion comprising the 3′ terminal region, wherein the second portion is hybridized to a first haplomer. The first haplomer comprises a hybridization region that is hybridized to the second portion of the first nucleic acid aptamer, and a reactive effector moiety. The second nucleic acid aptamer comprises a first portion folded into a tertiary structure that is able to bind to a target molecule, and a second portion comprising the 5′ terminal region, wherein the second portion is hybridized to a second haplomer. The second haplomer comprises a hybridization region that is hybridized to the second portion of the second nucleic acid aptamer, and a reactive effector moiety. The reactive effector moiety of the first haplomer is capable of interacting with the reactive effector moiety of the second haplomer.

The present disclosure also provides binary nucleic acid aptamers comprising a first portion folded into a tertiary structure that is able to bind to a target molecule, a second portion folded into a tertiary structure that is able to bind to a target molecule and a third portion located between the first and second portion, wherein the third portion is hybridized to a first haplomer and a second haplomer. The first haplomer comprises a hybridization region that is hybridized to the third portion of the binary nucleic acid aptamer, and a reactive effector moiety. The second haplomer comprises a hybridization region that is hybridized to the third portion of the binary nucleic acid aptamer, and a reactive effector moiety. The reactive effector moiety of the first haplomer is in spatial proximity to the reactive effector moiety of the second haplomer.

The present disclosure also provides aptamers comprising the nucleotide sequence set forth in SEQ ID NO:1, SEQ ID NO:2, SEQ ID NO:3, or SEQ ID NO:4.

The present disclosure also provides methods of selecting an aptamer from a library comprising: binding of members of the library to a desired solid phase target; washing the solid phase target; eluting the bound members of the library; precipitating the bound members of the library; reconstituting the bound members of the library; testing the bound members of the library for the best amplifiable concentration; performing preparative asymmetric PCR; testing the PCR products on a gel; binding the PCR products to streptavidin magnetic beads; washing the streptavidin magnetic beads; eluting the top strands; testing the eluted strands on a gel; and performing the cycle nine to ten times until diversity of the binding aptamer population is sufficiently reduced such that analysis of the binding properties of specific predominant aptamer clones can be performed.

The present disclosure also provides methods of selecting an aptamer having an accessible 3′ or 5′ terminal end for hybridization to a haplomer comprising: contacting an aptamer with a corresponding target molecule; contacting the aptamer with an oligonucleotide probe having a region that is complementary to the 3′ or 5′ terminal end of the aptamer, wherein the oligonucleotide probe is conjugated to biotin; washing the aptamer-oligonucleotide probe complex to remove unbound oligonucleotide probe; contacting the aptamer-oligonucleotide probe complex with streptavidin magnetic beads; washing the streptavidin magnetic beads and eluting the aptamer, wherein the aptamer possesses an accessible 3′ or 5′ terminal end for hybridization to a haplomer.

The present disclosure also provides methods of preparing a binary aptamer comprising: contacting a target molecule or target cell with a plurality of aptamers; eluting the bound aptamers, including at least one left aptamer and at least one right aptamer; contacting the target molecule or target cell with the population of bound left and right aptamers; contacting the bound aptamers with a ligase and an RNA splint; removing the splint with RNase H; thereby resulting in a covalently ligated binary aptamer.

The present disclosure also provides methods of delivering at least one aptamer to a pathogenic cell, said method comprising: administering a therapeutically effective amount of any one or more aptamers of any one of claims 1 to 18 to the pathogenic cell, wherein at least one active effector structure in the pathogenic cell is produced.

### BRIEF DESCRIPTION OF THE DRAWINGS

FIG. 1 shows a representative method for protein or other macromolecular targets.

FIG. 2 shows a representative deployment of aptamers for macromolecular templating, whereby regions of aptamer sequences are used for hybridization-based templating of effector partial moieties bearing bioorthogonal reactive groups.

FIG. 3 shows representative dual aptamer library configurations (N=random region, where N > or equal to 40; P=5′ phosphate group; Pr-a, Pr-b=primer a and b sequences, respectively, for Left-aptamers; Pr-c, Pr-d=primer c and d sequences, respectively, for Right-aptamers; TL, TR=Left and Right template regions respectively.

FIG. 4 shows a representative aptamer selection process for each single library (“singlet” aptamers).

FIG. 5 shows a representative use of differential strand biotinylation to prepare large amounts of single-stranded aptamer selected subpopulations.

FIG. 6 shows a representative asymmetric PCR process for generating single-strands during aptamer selection cycle.

FIG. 7 (panels A and B) shows a representative templating effector partial reactivity by single aptamers at either the 3′ ends (panel A) or 5′ ends (panel B).

FIG. 8 shows a representative selection of singlet aptamers binding a specific target with an accessible terminal sequence for hybridization, with a biotinylated probe oligonucleotide.

FIG. 9 (panels A and B) shows a representative templating effector partial reactivity by dual proximal aptamers (panel A) or ligated binary pairs (panel B).

FIG. 10 shows a representative binary aptamer selection involving co-binding on a target molecule.

FIG. 11 shows a representative general binary aptamer structure (L, R=Left and Right aptamer sequences, respectively.

FIG. 12 shows a representative formation of a binary aptamer from a pair of aptamers co-binding proximally close target sites on a complex molecule.

FIG. 13 shows a representative accelerated selection process for a binary aptamer.

FIG. 14 shows a representative accelerated selection process for a binary aptamer.

FIG. 15 shows a representative schematic of appending an unnatural L-DNA tag onto the 5′ end of a singlet aptamer.

FIG. 16 shows a representative schematic of appending an unnatural L-DNA tag onto the 3′ end of a singlet aptamer.

FIG. 17 shows a representative schematic of appending unnatural L-DNA tags onto the 3′ and 5′ ends of dual aptamers, for directing spatial proximity of effector partial moieties by bioorthogonal hybridization.

FIG. 18 (panels A and B) shows a representative schematic for equipping binary aptamers (panel A=left aptamer; panel B=right aptamer) with bridging unnatural L-DNA sequences, for directing spatial proximity of effector partial moieties by bioorthogonal hybridization.

FIG. 19 shows another representative schematic for equipping binary aptamers with bridging unnatural L-DNA sequences, for directing spatial proximity of effector partials by bioorthogonal hybridization.

FIG. 20 shows a representative selection process for aptamer allostery, where target binding induces the exposure/accessibility of the template sequence.

FIG. 21 shows a representative application of aptamer allostery towards the in situ generation of joined binary aptamers.

FIG. 22 shows a representative subtraction between aptamers binding targets from a tumor cell source and those binding a matched cognate normal cell.

FIG. 23 shows another representative subtraction between aptamers binding targets from a tumor cell source and those binding a matched cognate normal cell.

FIG. 24 (panels A and B) show representative N-terminal extracellular domain of the human MC1R protein, and co-binding experiments with both combinations of L- and R-aptamer subpopulations binding pentapeptides, respectively.

FIG. 25 shows a representative cycle analyses of aptamers.

FIG. 26 shows a representative demonstration of successful co-binding after 4 cycles of Fab selection, and sequence analysis of a co-binding experiment binary aptamer product.

FIG. 27 shows a representative cycle analyses of aptamers binding biotinylated Fab.

FIG. 28 shows a representative co-binding test of a 10th cycle of L- and R-aptamers to biotinylated Fab.

FIG. 29 shows a representative binary clone 10CB-01 obtained from 10th cycle co-binding experiment.

FIG. 30 shows a representative direct binding of specific 10^{th} cycle aptamers to “No biotinylated Fab” (bFab).

FIG. 31 shows a representative binding of biotinylated Fab target by binary form of known Fab-binding singlet aptamers.

FIG. 32 shows a representative demonstration of co-binding on IgG1 target of 10th cycle Fab-selected aptamers.

FIG. 33 shows representative sequences of binary aptamer junctions and test aptamer template-directed ligation oligonucleotides.

FIG. 34 shows a representative structure of trans-cyclooctene (TCO) ester and methyltetrazine (MTZ) ester reagents for amino-terminal oligonucleotide derivatization.

FIG. 35 shows a representative annealing of click-labeled oligonucleotides to a target molecule template, resulting in the spatial proximity of duplexed ends with compatible restriction site overhangs.

FIG. 36 shows a representative schematic for solid-phase oligonucleotide-based templating in aptamers.

FIG. 37 shows a representative demonstration of PCR product formation for end-joined oligonucleotides in situ on solid-phase streptavidin magnetic beads.

FIG. 38 shows a representative demonstration of templating of model inverse electron-demand Diels-Alder (IEDDA) click reactions by aptamers templates while bound to bFab target in situ.

FIG. 39 shows a representative PCR testing of binding and binary formation of aptamers bound to bFab in situ.

FIG. 40 (panels A, B, and C) show a representative formation and testing of binary aptamers in situ on bFab target.

FIG. 41 shows a representative schematic of alternative in situ formation of template from a proximal binary aptamer pair by means of a short stem loop bridge.

FIG. 42 shows a representative structure of aptamers used for testing the complementary-end stem loop bridge binary templating approach, and corresponding sequences.

FIG. 43 shows a representative testing of the effect of aptamer extensions on the ability to bind bFab-BRD7.

FIG. 44 (panels A and B) shows a representative testing of the complementary-end binary stem-loop aptamer approach.

FIG. 45 shows a representative binding curve for aptamer #228 and biotinylated Fab-BRD7.

FIG. 46 shows representative first steps in forming cell-surface complexes for demonstrating aptamer-mediated peptide assembly via templated assembly.

FIG. 47 shows representative remaining steps in forming cell-surface complexes for demonstrating aptamer-mediated peptide assembly via templated assembly, and a representative process of functional read-out by means of specific T cell receptor recognition.

### DESCRIPTION OF EMBODIMENTS

Cell-specific transcripts can be potentially exploited as templates for the directed assembly of novel structures capable of directly or indirectly killing the host cell. In particular, tumor-specific transcripts can be potentially exploited as templates for the directed assembly of novel structures capable of directly or indirectly killing the tumor host. This technology would be greatly extended if non-RNA molecules could be used to template assembly reactions in a cell-type specific manner, particularly if it was possible to exploit surface target molecules in this manner. The latter capability would have great benefit through the reduction of logistical delivery problems. In principle, both of these desirable features can be fulfilled through the use of nucleic acid aptamers, where the aptamers act in a dual role as both recognition agents towards the target of interest, and provide an accessible nucleic acid sequence upon which templated assembly can be performed.

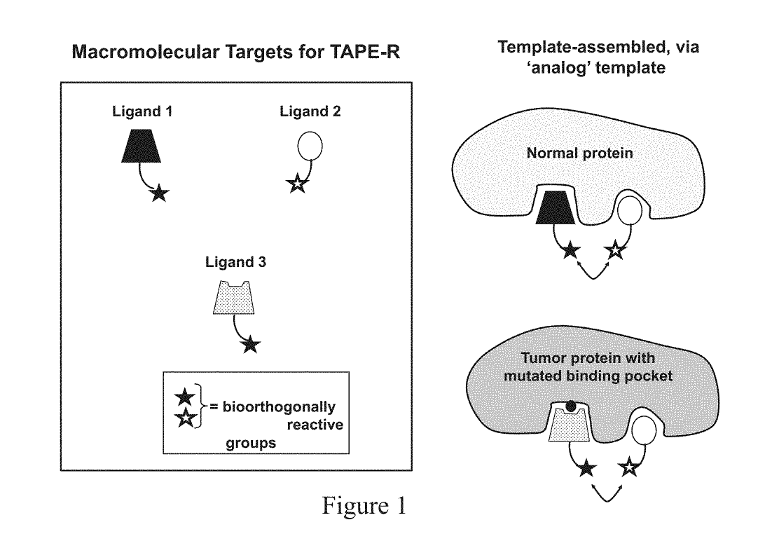

In principle, a pair of ligands (i.e., partial effector moieties as previously described in, for example, PCT International Publication WO 14/197547; now referred to herein as “haplomers”) covalently carrying reactive effector moieties (i.e., combinable portions of a desired effector product) can complete effector product assembly upon any templating structure, provided that the template-ligand (i.e., aptamer-haplomer) binding results in the necessary spatial proximity for mutual reactivity between two reactive effector moieties to occur (see, FIG. 1). Accordingly, other molecules beyond nucleic acids can, in principle, act as guides for specific templated assembly processes. Such non-nucleic acid templates may include proteins and complex carbohydrates, either alone or in combination. Also, either proteins or complex carbohydrates can, in principle, act as templates in concert with nucleic acids, where each are present within specific ribonucleoproteins, with or without glycosyl modifications.

An approach where few assumptions are made as to the nature of analog templating sites uses nucleic acid aptamers. Here aptamers are selected as ligands themselves for proteomic/glycomic/nucleic acid targets, and those binding to targets in spatial proximity are potentially useful as carriers of haplomers for templated assembly. Pairs of aptamers can be used as such carrier ligands, or alternatively a single selected aptamer can be used in concert with a known ligand, also carrying a haplomer.

Since aptamers can be selected to bind to non-nucleic acid target molecules expressed on cell surfaces, they are particularly useful for recognition and adaptive templating of novel surface structures found on specific cells, such as tumor cells. However, since most aptamers are not large nucleic acid molecules (i.e., many are less than 100 bases), and may often assume a folded and compacted structure, they are more readily transfected into target cells than many protein-based reagents. Thus, intracellular targets for aptamers are also desired. Such intracellular targets can also include RNA molecules, particularly where the RNA exists in a well-folded stable state. The latter configurations may often be refractory to conventional hybridization-mediated templated assembly, but amenable to recognition and secondary adaptive templating by aptamers.

Aptamers are single-stranded folded nucleic acids which have been selected for the ability to bind to a specific target molecule of interest. In some embodiments, the selection process involves the synthesis of a nucleic acid molecule with an extended random tract flanked on the 3′ or 5′ terminal end by specific primer sequences which enable amplification of the random population, or any members thereof with specific sequences. Within a large random population, a library of structural motifs arising from self-folding of the random region is generated and, in principle, a wide range of target molecules can be bound by specific members of this library. These specific binding nucleic acid molecules can be enriched by appropriate selection procedures, and then amplified. After such amplification of the initially very small subset of nucleic acid molecules that bind a desired target molecule, the selection round is repeated, promoting further enrichment of the desired nucleic acid molecules. In addition, this cycle is evolutionary, since mutations arising during the amplification process which enhance binding are favored and, after sufficient repetitions, specific nucleic acid molecules which bind with high affinity to the desired target molecule of interest can be isolated and identified. Such specific nucleic acid molecules that bind to the desired target molecule of interest with high affinity serve as nucleic acid aptamers, which in turn can serve as templates for templated assembly of functional products that can modify a cell.

In general, since aptamers are composed of nucleic acids, they can potentially provide a short linear sequence for templating purposes, as a contiguous segment of their primary sequences. Such a “built-in” templating sequence can, in principle, be located anywhere within the primary aptamer sequence, provided that hybridization of haplomers onto the aptamer does not disrupt binding of the aptamer to the target molecule of interest. In practice, though, targeting of either 5′ or 3′ terminal regions of aptamer sequences is likely to have a lower probability of disrupting aptamer function. Such terminal sites are easier to modify as desired, or to generate as secondarily appended segments.

In any of the aptamers described herein (including those with or without their hybridized haplomer(s)), the nucleic acid can comprise DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), XNA, morpholino nucleic acid analogues (morpholinos), pseudouridine nucleotides, xanthine nucleotides, hypoxanthine nucleotides, 2-deoxyinosine nucleotides, or other nucleic acid analogues capable of base-pair formation, or any combination thereof. In some embodiments, the nucleic acid is or comprises a portion which is LNA. In some embodiments, in any of the nucleic acid aptamers described herein, the hybridization region of the haplomer and the portion of the aptamer which hybridizes to the hybridization region of the haplomer both comprise L-DNA. In addition, aptamers can be very flexible. For example, aptamers can be modified such that nuclease resistance is conferred by means of modified backbones, including, but not limited to, phosphorothioates, or 2′ modifications, including, but not limited to, 2′-O-methyl derivatives. Alternately, L-DNA analogs (spiegelmers) binding desired targets can be used where applicable, and have high nuclease resistance.

The present disclosure provides nucleic acid aptamers. In some embodiments, the aptamer is a singlet nucleic acid aptamer. The singlet nucleic acid aptamer comprises a first portion that is folded into a tertiary structure that is able to bind to a target molecule. This first portion of the singlet aptamer that is folded into a tertiary structure can comprises either the 3′ or 5′ terminal region of the aptamer. The singlet nucleic acid aptamer also comprises a second portion that comprises either the 3′ or 5′ terminal region of the aptamer (i.e., whichever terminal region is not a part of the first portion). Thus, in some embodiments, the first portion that is folded into a tertiary structure that is able to bind to a target molecule comprises the 5′ portion of the aptamer, leaving the second portion to comprise the 3′ terminal region of the aptamer. Alternately, the first portion that is folded into a tertiary structure that is able to bind to a target molecule can comprises the 3′ portion of the aptamer, leaving the second portion to comprise the 5′ terminal region of the aptamer.

Both the first portion and second portion of the singlet aptamer comprise sequence regions that serve as primer binding sites for amplification purposes. In some embodiments, the 5′ terminal region of the aptamer contains a first sequence region that serves as a first primer binding site for amplification purposes. In some embodiments, the 3′ terminal region of the aptamer contains a second sequence region that serves as a second primer binding site for amplification purposes. Using both amplification primer binding sites in conjunction with the appropriate primers allows for amplification, such as by PCR, of the singlet aptamer. In some embodiments, the respective primer binding regions in the second portion of the singlet aptamer can also form part of the template regions for producing the functional product upon template assembly.

In some embodiments, the second portion of the singlet nucleic acid aptamer is hybridized to a first haplomer and a second haplomer. When an aptamer is hybridized to a first haplomer and/or a second haplomer, the complex thus formed is termed herein the “aptamer-haplomer” complex. In some embodiments, the second portion of the singlet nucleic acid aptamer is not hybridized to the first haplomer and/or the second haplomer. In either case, the second portion of the singlet nucleic acid aptamer comprises a region that is complementary to at least one portion of the first haplomer and comprises a region that is complementary to at least one portion of the second haplomer. Thus, in some embodiments, the aptamer lacks the first haplomer and/or the second haplomer hybridized thereto. Accordingly, in other embodiments, the aptamer includes the first haplomer and/or the second haplomer hybridized thereto.

The first haplomer comprises a hybridization region that is able to hybridize (i.e., the hybridization region of the haplomer is sufficiently complementary) to the second portion of the singlet nucleic acid aptamer. The first haplomer also comprises a reactive effector moiety. The second haplomer also comprises a hybridization region that is able to hybridize (i.e., the hybridization region of the haplomer is sufficiently complementary) to the second portion of the singlet nucleic acid aptamer. The second haplomer also comprises a reactive effector moiety.

The reactive effector moiety of the first haplomer is in spatial proximity to the reactive effector moiety of the second haplomer. The reactive effector moiety of the first haplomer and the reactive effector moiety of the second haplomer are in spatial proximity when a chemical reaction (such as any one of the chemical reactions described below) can occur between the respective template reactive effector moieties such that the two reactive effector moieties are joined to form a single functional product.

In some embodiments, the aptamer is a dual proximal nucleic acid aptamer pair. The first nucleic acid aptamer of the dual proximal nucleic acid aptamer pair comprises a first 5′ portion that is folded into a tertiary structure that is able to bind to a desired target molecule. The first nucleic acid aptamer of the dual proximal nucleic acid aptamer pair also comprises a second portion comprising the 3′ terminal region. In some embodiments, the first aptamer of the dual proximal aptamer pair is termed the “left” aptamer.

In some embodiments, the second portion of the first nucleic acid aptamer of the dual proximal nucleic acid aptamer pair is hybridized to a first haplomer. When a first aptamer is hybridized to a first haplomer, the complex thus formed is termed herein the “aptamer-haplomer” complex. In some embodiments, the second portion of the first nucleic acid aptamer of the dual proximal nucleic acid aptamer pair is not hybridized to the first haplomer. In either case, the second portion of the first nucleic acid aptamer of the dual proximal nucleic acid aptamer pair comprises a region that is complementary to at least one portion of the first haplomer. Thus, in some embodiments, the first nucleic acid aptamer of the dual proximal nucleic acid aptamer pair lacks the first haplomer hybridized thereto, and in other embodiments, the first nucleic acid aptamer of the dual proximal nucleic acid aptamer pair includes the first haplomer hybridized thereto.

The first haplomer comprises a hybridization region that is able to hybridize (i.e., the hybridization region of the haplomer is sufficiently complementary) to the second portion of the first nucleic acid aptamer of the dual proximal nucleic acid aptamer pair. The first haplomer also comprises a reactive effector moiety. This first haplomer is analogous to the first haplomer for the singlet aptamer described above.

The second nucleic acid aptamer of the dual proximal nucleic acid aptamer pair comprises a first 3′ portion that is folded into a tertiary structure that is able to bind to a desired target molecule. The second nucleic acid aptamer of the dual proximal nucleic acid aptamer pair also comprises a second portion comprising the 5′ terminal region. In some embodiments, the second aptamer of the dual proximal aptamer pair is termed the “right” aptamer.

In some embodiments, the second portion of the second nucleic acid aptamer of the dual proximal nucleic acid aptamer pair is hybridized to a second haplomer. When a second aptamer is hybridized to a second haplomer, the complex thus formed is termed herein the “aptamer-haplomer” complex. In some embodiments, the second portion of the second nucleic acid aptamer of the dual proximal nucleic acid aptamer pair is not hybridized to a second haplomer. In either case, the second portion of the second nucleic acid aptamer of the dual proximal nucleic acid aptamer pair comprises a region that is complementary to at least one portion of the second haplomer. Thus, in some embodiments, the second nucleic acid aptamer of the dual proximal nucleic acid aptamer pair lacks the second haplomer hybridized thereto, and in other embodiments, the second nucleic acid aptamer of the dual proximal nucleic acid aptamer pair includes the second haplomer hybridized thereto.

Similar to the first haplomer, the second haplomer comprises a hybridization region that is able to hybridize (i.e., the hybridization region of the haplomer is sufficiently complementary) to the second portion of the second nucleic acid aptamer of the dual proximal nucleic acid aptamer pair. The second haplomer also comprises a reactive effector moiety. This second haplomer is analogous to the second haplomer for the singlet aptamer described above.

The reactive effector moiety of the first haplomer and the reactive effector moiety of the second haplomer are in spatial proximity when a chemical reaction (such as any of the chemical reactions described below) can occur between the respective haplomers such that the two template reactive effector moieties are joined to form a single functional product.

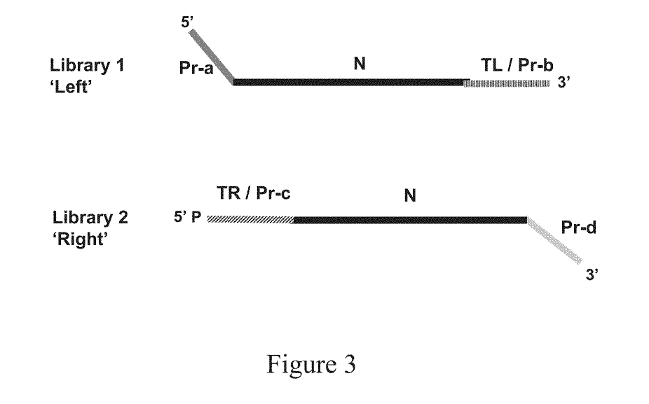

FIG. 3 shows representative dual aptamer library configurations. “Library 1 ‘Left’” refers to the first nucleic acid aptamer of the dual proximal nucleic acid aptamer pair described herein and “Library 2 ‘Right’” refers to the second nucleic acid aptamer of the dual proximal nucleic acid aptamer pair described herein. Each of these two haplomers generally corresponds to the singlet aptamers described herein, except that for a singlet aptamer, the first and second haplomers bind to the same singlet aptamer, whereas for a dual proximal aptamer pair, the first haplomer binds to the first (i.e., left) aptamer and the second haplomer binds to the second (i.e., right) aptamer. Also referring to FIG. 3, “P” represents a 5′ phosphate group.

The portions of the aptamers, referring again to FIG. 3, labeled as “Pr-a”, “Pr-b”, “Pr-c”, and “Pr-d” are the primer binding sites for amplification of the particular aptamer. These sites correspond to the sequence regions of the singlet aptamer described above that serve as primer binding sites for amplification purposes.

The portions of the aptamers, referring again to FIG. 3, labeled as “TL” and “TR” are the regions of the aptamer that hybridize to the haplomers. In some embodiments, one or both of “TL” and “TR” can include a portion of either or both of the adjacent “N” region and adjacent primer region. The “TL” and “TR” regions of the dual proximal aptamers correspond to the second portion that comprises either the 3′ or 5′ terminal region of the singlet aptamer (i.e., whichever terminal region is not a part of the first portion of the singlet aptamer).

Continuing to refer to FIG. 3, “N” represents an initial random nucleotide region. In some embodiments, “N” is from about 20 nucleotides to about 60 nucleotides. In some embodiments, N is greater than or equal to 40 nucleotides. Each of the aptamers described herein comprises the “N” region. That is, the “N” region depicted in FIG. 3 corresponds to the first portion of the singlet aptamer that is folded into a tertiary structure that is able to bind to a desired target molecule.

The “N” region is randomized such that the initial aptamer population comprises a very large number of molecules with variant sequences through this region, but with fixed primer sites. Some aptamers with specific sequences occurring within the large random population can fold in such a way that they can bind desired target molecules. These aptamers are progressively selected and amplified via the aptamer isolation process (often known as the SELEX process). Thus, the specific “N” sequences from the random region population are chief contributors to target binding via their tertiary folding (as set forth above). However, the primer sites adjacent to the “N” regions (but not the “Pr-b” and “Pr-c” regions depicted in FIG. 3) can also contribute to target binding or target-aptamer stabilization in some cases, depending on the details of the selection process.

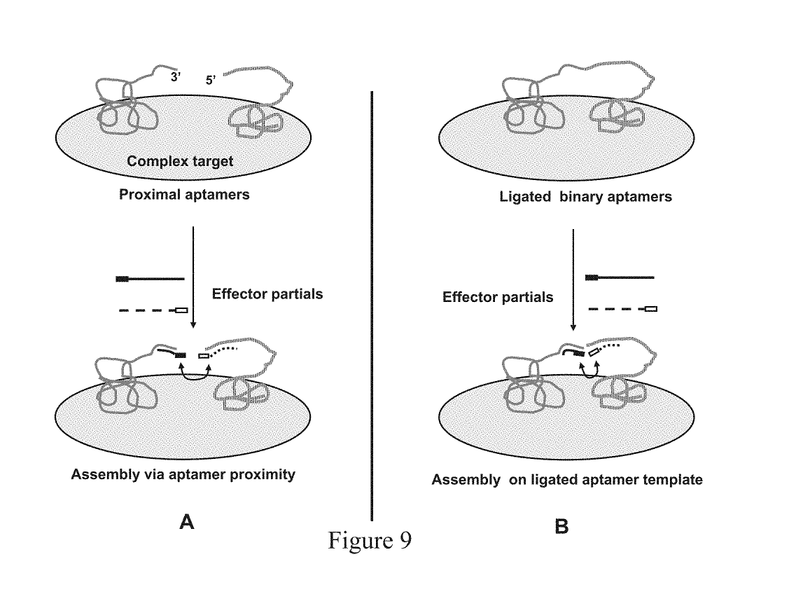

FIG. 9A shows representative dual proximal aptamers binding to a complex target (as in, for example, a cell-surface protein). The curved arrows denote a proximity-induced chemical reaction between different effector haplomers (i.e., “Effector partials”).

In some embodiments, the aptamer is a binary nucleic acid aptamer. The binary nucleic acid aptamer comprises a first portion (i.e., left portion) that is folded into a tertiary structure that is able to bind to a desired target molecule. The first portion also comprises a primer biding region on the 5′ terminal end for amplification purposes. The binary nucleic acid aptamer also comprises a second portion (i.e., right portion) that is also folded into a tertiary structure that is also able to bind to a desired target molecule. The second portion also comprises a primer biding region on the 3′ terminal end for amplification purposes. The binary nucleic acid aptamer also comprises a third portion that is located between the first portion and the second portion. In some embodiments, the 3′ terminal end of a first aptamer (i.e., left aptamer of a dual proximity aptamer pair) and 5′ terminal end of a second aptamer (i.e., right aptamer of a dual proximity aptamer pair) can be ligated together to form the binary aptamer. For example, referring to the dual proximal nucleic acid aptamer pair, the 5′ and 3′ terminal ends of the aptamer pair can be ligated together. In some embodiments, the first portion of the binary aptamer is termed the “left” aptamer and the second portion of the binary aptamer is termed the “right” aptamer.

In some embodiments, the first portion of the binary nucleic acid aptamer that is folded into a tertiary structure that is able to bind to a target molecule and the second portion that is also folded into a tertiary structure that is able to bind to a target molecule each, independently, have a Tₘ from about 45° to about 85° C., from about 45° to about 80° C., from about 45° to about 75° C., from about 50° to about 70° C., from about 50° to about 65° C., from about 55° to about 70° C., or from about 55° to about 65° C.

The third portion of the binary aptamer comprises the region to which the haplomers are hybridized, as described further below. The third portion of the binary aptamer also comprises the 3′ primer biding region for application of the first portion (along with the 5′ primer binding region of the first portion) and the 5′ primer binding region for amplification of the second portion (along with the 3′ primer binding region of the second portion). In some embodiments, the binary aptamer selection process requires that the third portion be accessible to hybridizing splint molecules, consequently selecting against aptamers that require the presence of these regions for their binding to target. Only aptamers that bind to target with free (accessible) third portions are, thus, theoretically selectable by the binary selection process. In other words, as a result of the singlet-to-binary selection process, the third portion available for the templating process are not likely to significantly contribute to target binding.

In some embodiments, the third portion of the binary nucleic acid aptamer is hybridized to a first haplomer and a second haplomer. In some embodiments, the third portion of the binary nucleic acid aptamer pair is not hybridized to the first haplomer and/or the second haplomer. In either case, the third portion of the binary nucleic acid aptamer comprises a region that is complementary to at least one portion of the first haplomer and/or the second haplomer. Thus, in some embodiments, the binary nucleic acid aptamer lacks the first haplomer and/or the second haplomer, and in other embodiments, the binary nucleic acid aptamer includes the first haplomer and/or the second haplomer hybridized thereto.

The first haplomer (i.e., the left haplomer) comprises a hybridization region that is able to hybridize (i.e., the hybridization region of the haplomer is sufficiently complementary) to the third portion of the binary nucleic acid aptamer. The first haplomer also comprises a reactive effector moiety. The second haplomer (i.e., the right haplomer) also comprises a hybridization region that is able to hybridize (i.e., the hybridization region of the haplomer is sufficiently complementary) to the third portion of the binary nucleic acid aptamer. The second haplomer also comprises a reactive effector moiety.

The reactive effector moiety of the first haplomer and the reactive effector moiety of the second haplomer are in spatial proximity when a chemical reaction (such as any of the chemical reactions described below) can occur between the respective haplomers such that the two template reactive effector moieties are joined to form a single functional product.

In some embodiments, the third portion of the binary nucleic acid aptamer located between the first portion and the second portion (sometimes referred to herein as the “splint region”) comprises from about 20 nucleotides to about 80 nucleotides in length, from about 30 nucleotides to about 70 nucleotides in length, from about 35 nucleotides to about 65 nucleotides in length, or from about 40 nucleotides to about 60 nucleotides in length.

In some embodiments, the third portion of the binary nucleic acid aptamer that is located between the first portion and the second portion comprises from about 10 nucleotides to about 90 nucleotides in length, from about 15 nucleotides to about 85 nucleotides in length, from about 20 nucleotides to about 80 nucleotides in length, or from about 25 nucleotides to about 75 nucleotides in length. In some embodiments, the third portion can be fully random (i.e., 25:25:25:25 dA:dC:dG:dT by synthetic ratios), or with any form of specific patterning, where defined bases are interspersed with random regions. As a non-limiting example, a random region of 61 bases designed to enhance selection of G Quadruplexes can take the form of (N₉-G₄)₄-N₉.

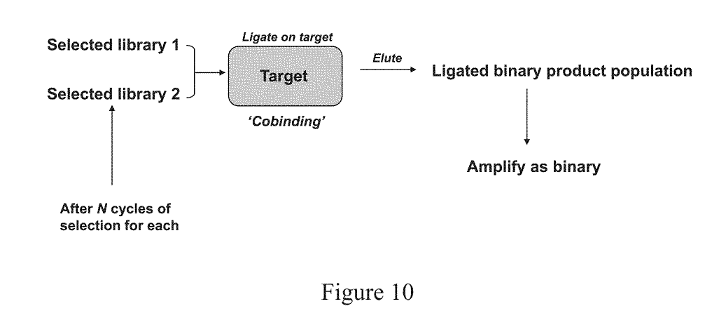

FIG. 9, Panel B shows representative binary aptamers binding to a complex target (as in, for example, a cell-surface protein). In addition, as described further herein, binary aptamers can be formed from dual proximal aptamer pairs that bind a target molecule in suitable spatial proximity, in a process herein termed “co-binding”, followed by ligation (see, for example FIG. 10). Depending on the specific parameters of aptamer spacing, binary aptamers providing a contiguous template for haplomers may be more efficient than dual proximal aptamers. Referring to FIG. 9, the curved arrows denote proximity-induced reaction between different effector partial moieties.

The target molecule(s), to which one or more of the nucleic acid aptamers bind, can be any protein or post-translationally modified protein, protein complex, carbohydrate, lipid, phospholipid, glycolipid, nucleic acid, or ribonucleoprotein associated with a cell. Particular target molecules include, but are not limited to, surface-expressed molecules, general and intracellular proteins, carbohydrates, lipid-related molecules, and nucleic acid molecules. Surface-expressed molecules include, but are not limited to: 1) integrins (such as, for example, integrin-β1); 2) melanocortin-1 receptor (MC1R); 3) other G-Protein coupled receptors (GPCRs); 4) immune cell markers (such as, for example, IgM, IgA, IgG, IgE (all isotypes), MHC Class I and Class II molecules, CD19, CD20, CD27, CD28, CTLA-4, and PD-1); 5) phosphatidylserine; 6) phosphatidylethanolamine; and 7) growth factor receptors (such as, for example, HER-2/neu and EGFR). General and intracellular proteins include, but are not limited to: 1) kinases; 2) enzymes; 3) transcription factors; 4) post-translationally modified proteins; 5) mutated proteins; and 6) protein complexes. Carbohydrates include, but are not limited to, complex carbohydrates appended to proteins (glycoproteins) or other molecules as carriers. Lipid-related molecules include, but are not limited to phospholipids and glycolipids. Nucleic acid molecules include, but are not limited to ribonucleoproteins and mRNA structural motifs.

In some embodiments, when more than one aptamer is being used, the aptamers can bind to the same target molecule such that the aptamers are in physical proximity. For example, referring to the dual proximal nucleic acid aptamer pair, the aptamer pair may bind to the same target molecule such that the aptamer pair is in physical proximity. Alternately, the aptamer pair can bind to different target molecules on the same cell such that the aptamers are in physical proximity. In addition, the aptamer pair can bind to different target molecules on different cells such that the aptamers are in physical proximity. In some embodiments, the target molecule can be intracellular. Alternately, the target molecule can be on a cell surface.

In some embodiments, referring to any of the nucleic acid aptamers described herein, the hybridization region of the first haplomer and/or the second haplomer independently comprises from about 6 to about 24 nucleotides in length, from about 8 to about 20 nucleotides in length, or from about 10 to about 18 nucleotides in length.

In some embodiments, the terminal end of the first haplomer (comprising the reactive effector moiety) that is in spatial proximity with the terminal end of the second haplomer (comprising the reactive effector moiety) are covalently joined. In some embodiments, the first haplomer and the second haplomer are covalently joined to their respective ends in spatial proximity by a chemical reaction occurring between their respective reactive effector moieties. Numerous reactive effector moieties are disclosed in, for example, PCT International Publication WO 14/197547, which is incorporated herein by reference in its entirety.

The combination of two reactive effector moieties allows the formation of a functional product. The interaction between two reactive effector moieties can include physical interactions, such as chemical bonds (either directly linked or through intermediate structures), as well as non-physical interactions and attractive forces, such as electrostatic attraction, hydrogen bonding, and van der Waals/dispersion forces.

A reactive effector moiety can be biologically inert. In particular, the reactive effector moiety associated with a first haplomer can interact with a corresponding reactive effector moiety associated with a second haplomer, but will not readily interact with natural biomolecules. This is to ensure that the templated assembly product is formed only when corresponding effector partial moieties are assembled on an aptamer(s) bound to a target molecule. It also safeguards the reactive effector moiety from reacting with functional groups on other molecules present in the environment in which the assembly occurs, thus preventing the formation of unintended products. An example of a reactive effector moiety includes a bio-orthogonal moiety. A bio-orthogonal moiety reacts chemically with a corresponding bio-orthogonal moiety and does not readily react chemically with other biomolecules.

The reactive effector moiety provides a mechanism for templated reactions to occur in complex target compartments, such as a cell, virus, tissue, tumor, lysate, other biological structure, or spatial region within a sample that contains the target molecule, or that contains a different amount of target molecule than a non-target compartment. A reactive effector moiety can react with a corresponding reactive effector moiety, but does not react with common biochemical molecules under typical conditions. Unlike other reactive entities, the selectivity of reactive effector moiety prevents ablation of the reactive group prior to assembly of the product or reactant.

An example of a reactive effector moiety can include a bio-orthogonal moiety. The bio-orthogonal moiety can include those groups that can undergo “click” reactions between, for example, azides and alkynes, traceless or non-traceless Staudinger reactions between azides and phosphines, and native chemical ligation reactions between thioesters and thiols. Additionally, the bio-orthogonal moiety can be any of an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, a quadricyclane, and derivatives thereof.

Multiple reactive effector moieties can be used with the methods and compositions disclosed herein. Some non-limiting examples include the following.

Click chemistry is highly selective as neither azides nor alkynes react with common biomolecules under typical conditions. Azides of the form R—N₃ and terminal alkynes of the form R—C≡CH or internal alkynes of the form R—C≡C—R react readily with each other to produce Huisgen cycloaddition products in the form of 1,2,3-triazoles. Azides and azide derivatives may be readily prepared from commercially available reagents. Azides can also be introduced to a reactive effector moiety during synthesis of the reactive effector moiety. In some embodiments, an azide group is introduced into an reactive effector moiety comprised of a peptide by incorporation of a commercially available azide-derivatized standard amino acid or amino acid analogue during synthesis of the reactive effector moiety peptide using standard peptide synthesis methods. Amino acids may be derivatized with an azide replacing the α-amino group. Commercially available products can introduce azide functionality as an amino acid side chain, resulting in a structure of the form:

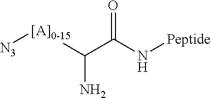

where A is any atom and its substituents in a side chain of a standard amino acid or non-standard amino acid analogue.

An azide may also be introduced into an reactive effector moiety peptide after synthesis by conversion of an amine group on the peptide to an azide by diazotransfer methods. Bioconjugate chemistry can also be used to join commercially available derivatized azides to chemical linkers, or reactive effector moieties that contain suitable reactive groups.

Standard alkynes can also be incorporated into reactive effector moieties by methods similar to azide incorporation. Alkyne-functionalized nucleotide analogues are commercially available, allowing alkyne groups to be directly incorporated at the time of reactive effector moiety synthesis. Similarly, alkyne-derivatized amino acid analogues may be incorporated into an reactive effector moiety by standard peptide synthesis methods. Additionally, diverse functionalized alkynes compatible with bioconjugate chemistry approaches may be used to facilitate the incorporation of alkynes to other moieties through suitable functional or side groups.

Standard azide-alkyne chemistry reactions typically require a catalyst, such as copper(I). Since copper(I) at catalytic concentrations is toxic to many biological systems, standard azide-alkyne chemistry reactions have limited uses in living cells. Copper-free click chemistry systems based on activated alkynes circumvent toxic catalysts. Activated alkynes often take the form of cyclooctynes, where incorporation into the cyclooctyl group introduces ring strain to the alkyne.

Heteroatoms or substituents may be introduced at various locations in the cyclooctyl ring, which may alter the reactivity of the alkyne or afford other alternative chemical properties in the compound. Various locations on the ring may also serve as attachment points for linking the cyclooctyne to a reactive effector moiety or linker. These locations on the ring or its substituents may optionally be further derivatized with accessory groups. Multiple cyclooctynes are commercially available, including several derivatized versions suitable for use with standard bioconjugation chemistry protocols. Commercially available cyclooctyne derivatized nucleotides can aid in facilitating convenient incorporation of the reactive effector moiety during nucleic acid synthesis.

The Staudinger reduction, based on the rapid reaction between an azide and a phosphine or phosphite with loss of N₂, also represents a bio-orthogonal reaction. The Staudinger ligation, in which covalent links are formed between the reactants in a Staudinger reaction, is suited for use in templated assembly. Both non-traceless and traceless forms of the Staudinger ligation allow for a diversity of options in the chemical structure of products formed in these reactions.

The standard Staudinger ligation is a non-traceless reaction between an azide and a phenyl-substituted phosphine such as triphenylphosphine, where an electrophilic trap substituent on the phosphine, such as a methyl ester, rearranges with the aza-ylide intermediate of the reaction to produce a ligation product linked by a phosphine oxide. Phenyl-substituted phosphines carrying electrophilic traps can also be readily synthesized. Derivatized versions are available commercially and suitable for incorporation into templated assembly reactants.

In some embodiments, phosphines capable of traceless Staudinger ligations can be utilized as reactive effector moieties. In a traceless reaction, the phosphine serves as a leaving group during rearrangement of the aza-ylide intermediate, creating a ligation typically in the form of a native amide bond. Compounds capable of traceless Staudinger ligation generally take the form of a thioester derivatized phosphine or an ester derivatized phosphine. Ester derivatized phosphines can also be used for traceless Staudinger ligation. Thioester derivatized phosphines can also be used for traceless Staudinger ligations.

Chemical linkers or accessory groups can optionally be appended as substituents providing attachment points for reactive effector moieties or for the introduction of additional functionality to the reactant.

Compared to the non-traceless Staudinger phenylphosphine compounds, the orientation of the electrophilic trap ester on a traceless phosphinophenol is reversed relative to the phenyl group. This enables traceless Staudinger ligations to occur in reactions with azides, generating a native amide bond in the product without inclusion of the phosphine oxide. The traceless Staudinger ligation may be performed in aqueous media without organic co-solvents if suitable hydrophilic groups, such as tertiary amines, are appended to the phenylphosphine. Preparation of water-soluble phosphinophenol, which can be loaded with a desired reactive effector moiety containing a carboxylic acid (such as the C-terminus of a peptide) via the mild Steglich esterification using a carbodiimide such as dicyclohexylcarbodiimide (DCC) or N,N′-diisopropylcarbodiimide (DIC) and an ester-activating agent such as 1-hydroxybenzotriazole (HOBT) has been reported (Weisbrod, et al., Synlett, 2010, 5, 787-789).

Phosphinomethanethiols represent an alternative to phosphinophenols for mediating traceless Staudinger ligation reactions. In general, phosphinomethanethiols possess favorable reaction kinetics compared with phosphinophenols in mediating traceless Staudinger reaction. U.S. Patent Publication 2010/0048866 and Tam et al., J. Am. Chem. Soc., 2007, 129, 11421-30 describe preparation of water-soluble phosphinomethanethiols. These compounds can be loaded with a peptide or other payload, in the form of an activated ester, to form a thioester suitable for use as a traceless bio-orthogonal reactive group.

Native chemical ligation is a bio-orthogonal approach based on the reaction between a thioester and a compound bearing a thiol and an amine. The classic native chemical ligation is between a peptide bearing a C-terminal thioester and another bearing an N-terminal cysteine. Native chemical ligation can be utilized to mediate traceless reactions producing a peptide or peptidomimetic containing an internal cysteine residue, or other thiol-containing residue if non-standard amino acids are utilized.

N-terminal cysteines can be incorporated by standard amino acid synthesis methods. Terminal thioesters can be generated by several methods known in the art, including condensation of activated esters with thiols using agents such as dicyclohexylcarbodiimide (DCC), or introduction during peptide synthesis via the use of “Safety-Catch” support resins.

Any suitable bio-orthogonal reaction chemistry can be utilized for synthesis of reactive effector moieties, as long as it efficiently mediates a reaction in a highly selective manner in complex biologic environments. A recently developed non-limiting example of an alternative bio-orthogonal chemistry that may be suitable is reaction between tetrazine and various alkenes such as norbornene and trans-cyclooctene, which efficiently mediates bio-orthogonal reactions in aqueous media.

Chemical linkers or accessory groups can optionally be appended as substituents to the above reactants, providing attachment points for nucleic acid moieties or for the introduction of additional functionality to the reactant.

The present disclosure also provides novel aptamers. In the aptamer sequences presented below, the first line provides the primer sequence (except where an underlined sequence is present, which denotes an extension for a stem-loop); the second line provides the 40-base sequence from the original random region; and the third line provides the other primer sequence (except where an underlined sequence is present, which denotes an extension for a stem-loop).

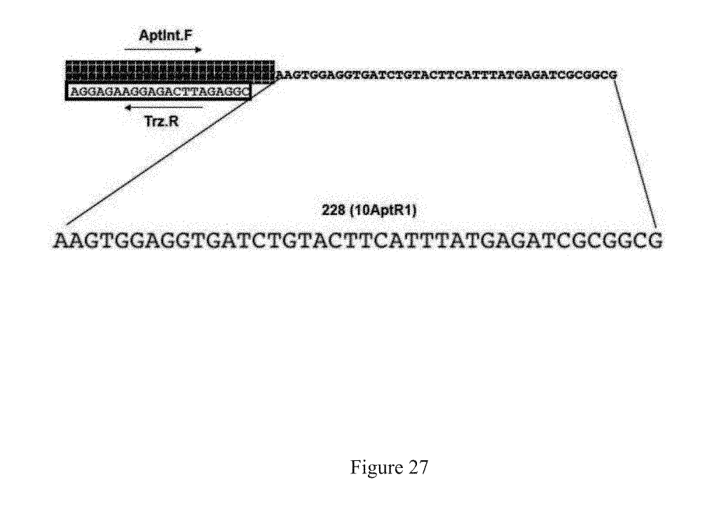

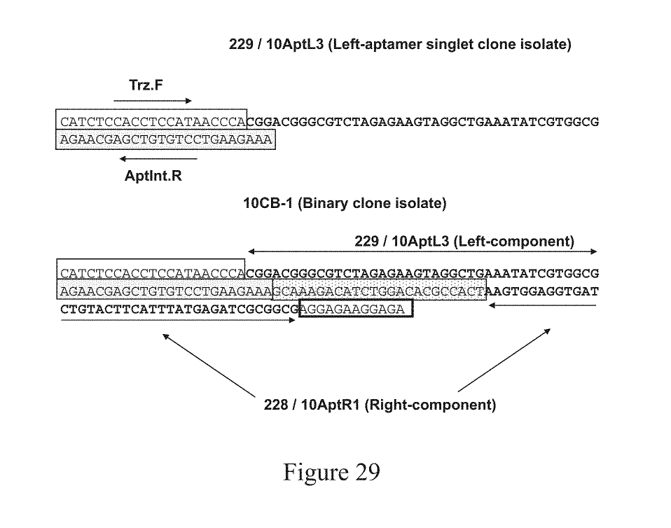

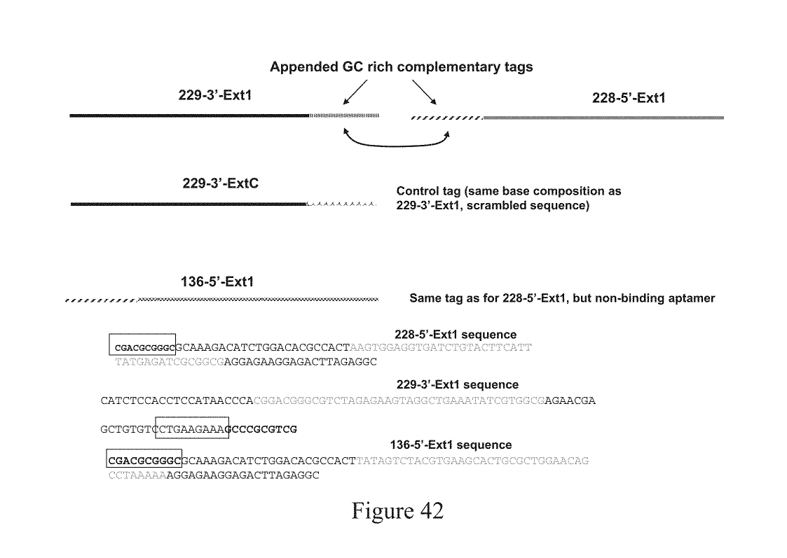

1) Aptamer 229 (10AptL3); 85-mer (see FIG. 29)
(SEQ ID NO: 1):
CATCTCCACCTCCATAACCCACGGACGGGCGTCTAGAGAAGTAGGCTGA
AATATCGTGGCGAGAACGAGCTGTGTCCTGAAGAAA
2) Aptamer 228 (10AptR1); 85-mer (see, FIG. 27)
(SEQ ID NO: 2):
GCAAAGACATCTGGACACGCCACTAAGTGGAGGTGATCTGTACTTCATT
TATGAGATCGCGGCGAGGAGAAGGAGACTTAGAGGC
3) Aptamer 229-3′-Ext1; 95-mer (see, FIG. 42)
(SEQ ID NO: 3):
CATCTCCACCTCCATAACCCACGGACGGGCGTCTAGAGAAGTAGGCTGA
AATATCGTGGCGAGAACGAGCTGTGTCCTGAAGAAACCGGCTGCGC
4) Aptamer 228-5′-Ext1; 95-mer (see, FIG. 42)
(SEQ ID NO: 4):
CGACGCGGGCGCAAAGACATCTGGACACGCCACTAAGTGGAGGTGATCT
GTACTTCATTTATGAGATCGCGGCGAGGAGAAGGAGACTTAGAGGC

Referring to FIG. 27, it has been observed that the primer site Trz.R helps the binding of Aptamer 228/10AptR1 to the target, and, likewise, the primer site Trz.F helps the binding of Aptamer 229/10AptL3 to the target.

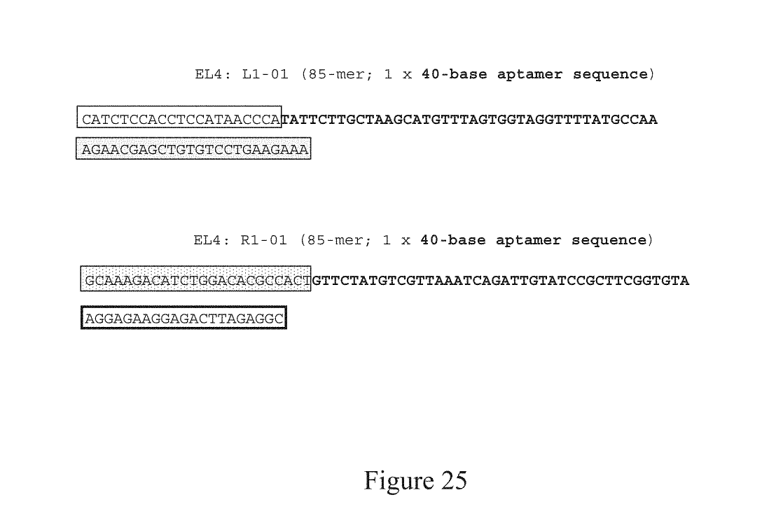

FIG. 25 shows a 4th cycle analyses of aptamers, in which arbitrary examples of cloned aptamers eluted from target Fab protein were obtained after 4 cycles of binding (EL4). L1-01 and R1-01, are arbitrary examples of Left- and Right aptamer clones, respectively. The bold sequences represent 40-mer tracts deriving from randomized sequence in the original aptamer libraries. The boxed sequences represent primer sites (no fill, primer Trz.F; light gray fill, primer AptInt.R (antisense in this orientation); speckled fill, primer AptInt.F; no fill, dark lines, primer Trz.R (antisense in this orientation)).

The present disclosure also provides populations of nucleic acid aptamers comprising two or more of the nucleic acid aptamers described herein. In some embodiments, the 5′ or 3′ ends of the aptamers are selected for accessibility to pairs of first and second haplomers.

The present disclosure also provides methods of selecting an aptamer from a library comprising, for example: binding members of the library to a desired solid phase target; washing the solid phase target; eluting the bound members of the library; precipitating the bound members of the library; reconstituting the bound members of the library; analyzing the bound members of the library for a suitable amplifiable concentration; performing preparative asymmetric PCR; testing the PCR products on a gel; binding the PCR products to streptavidin magnetic beads; washing the streptavidin magnetic beads; eluting the top strands; testing the eluted strands on a gel; and performing the cycle a plurality of times, such as up to nine or more times, until diversity of the binding aptamer population is sufficiently reduced such that analysis of the binding properties of specific predominant aptamer clones can be performed.

A general binding and elution procedure is described herein. In some embodiments, aptamers are initially prepared in standard phosphate-buffered saline with 1 mM magnesium chloride (PBSM), and heated for about 3 minutes at 80° C., followed by at least 5 minutes at 0° C. (ice bath) to allow for self-annealing and to minimize inter-aptamer interactions. In some embodiments, aptamer populations (or specific aptamers) are incubated with a target and rendered solid-phase.

In some embodiments, aptamer populations (or specific aptamers) can be incubated with a target for at least 1 hour at room temperature, then added to an excess of solid-phase capture matrix for greater than about 1 hour at room temperature. For primary aptamer populations, the initial incubation time with the target in solution is about 16 hours. For successive rounds of selected aptamer populations, the incubation time is about 2 to 4 hours. For specific aptamers, the incubation time is about 1 hour. Where the target is biotinylated, the capture matrix can be excess streptavidin magnetic beads (SAMBs) or any other streptavidin resin. Bead quantities can be calculated from the known molar input of biotinylated target and the maximal bead binding capacity data as provided by the manufacturer. SAMBs can be initially prepared by taking a predetermined volume in a storage buffer based on experimental needs, magnetically separating them, and washing twice with, for example, 1.0 ml of PBSM, using magnetic separation each time. Finally, the beads can be resuspended in the original volume of PBSM.

In other embodiments, the aptamer population (or specific aptamers) can be incubated with a target that has been previously rendered solid-phase on a suitable matrix. These matrices include, but are not limited to, streptavidin magnetic beads, streptavidin agarose, or any other streptavidin resin, where the target bears one or more biotin moieties. The target can also be covalently bound to the solid-phase matrix through various chemistries including, but not limited to, amine/N-hydroxy-succinimide, or thiol/maleimide. Such chemistries can covalently bind targets to magnetic beads or various other materials including, but not limited to, agarose or a polymerized resin.

In some embodiments, aptamers captured on solid-phase target matrix can be washed. The solid-phase matrices bearing targets and bound aptamers can be washed 1 time, 2 times, 3 times, or 4 times with, for example, 0.5 ml PBSM, with a final resuspension in the same volume of PBSM. Where SAMBs provide the solid-phase matrix, separations of matrix from supernatants during each wash cycle can be carried out by means of magnetic separation. Where other solid-phase materials are used, separations can be carried out by other means including, but not limited to, centrifugation or filtration.

In some embodiments, the bound aptamers are eluted. Aptamer/solid-phase target matrices can be separated from the final wash supernatants, as in the wash step above, and resuspended in, for example, about 100 μl of 0.1 M sodium hydroxide/5 mM EDTA for about 20 seconds at room temperature. The supernatant can be removed to a fresh tube, and the solid-phase material resuspended in, for example, about 100 μl of 0.1 M sodium hydroxide/5 mM EDTA for about 20 seconds at room temperature once more. Both supernatants can be pooled, and precipitated with, for example, about 20 μg of glycogen/20 μl of 3 M sodium acetate/600 μl ethanol for about 30 minutes at about −20° C. Preparations can be centrifuged (e.g., 10 minutes at maximum microfuge speed), and the pellets washed with, for example, 1 ml of 70% ethanol.

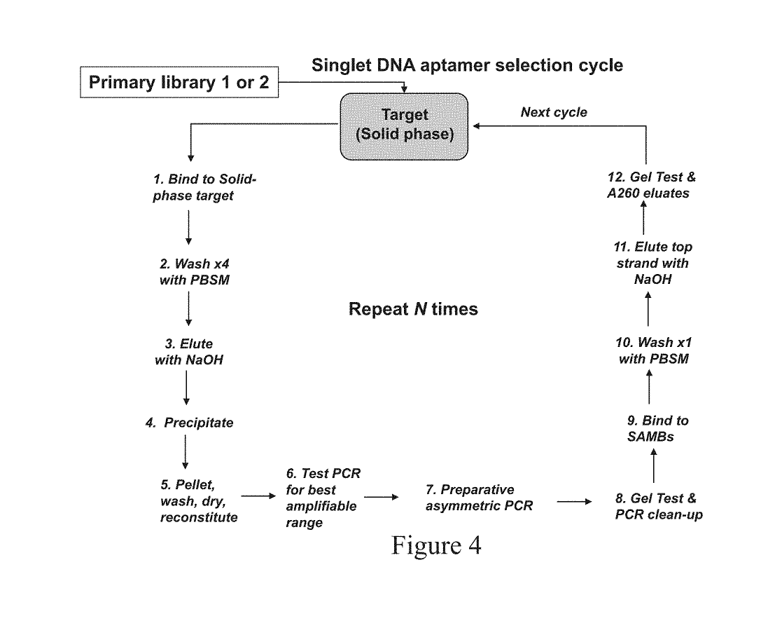

In some embodiments, the eluted aptamers can be reconstituted. For example, following the 70% wash from the step above, the preparations can be briefly centrifuged (e.g., for 1-2 minutes at maximum microfuge speed) and the supernatants removed. The resultant pellets can be dried and re-dissolved in an appropriate volume (e.g., usually 25 μl) of TE (10/1.0). Where the separation procedure uses magnetic beads, the resolubilized aptamer preparations can be subjected to another magnetic separation (e.g., 1 minute) to remove residual carry-over beads. Aptamer preparations can be quantitated spectrophotometrically at 260 nm, where an absorbance of 1.0=33 μg/ml single-stranded DNA. Samples can also be analyzed on, for example, 10% denaturing urea acrylamide gels. These preparations are termed herein primary eluted single-stranded aptamers for cycle N, where N is the number of times the cycling procedure has been repeated. FIG. 4 shows a representative aptamer selection process for each single library (e.g., “singlet” aptamers).

In some embodiments, the aptamers can be analyzed after about 9 to 10 cycles. For example, as the binding and elution cycles are continued, the proportion of the aptamer population that significantly binds to the target increases and, likewise, the diversity of the population (corresponding to variation in the N region of the first portion of a singlet aptamer, for example), commencing at maximal (i.e., random) diversity in the initial population) decreases. After about 9 to 10 cycles, typically clonal analysis of the aptamer population demonstrates recurrent independent clones with identical or related sequences, which correspond to population members with significant binding properties.

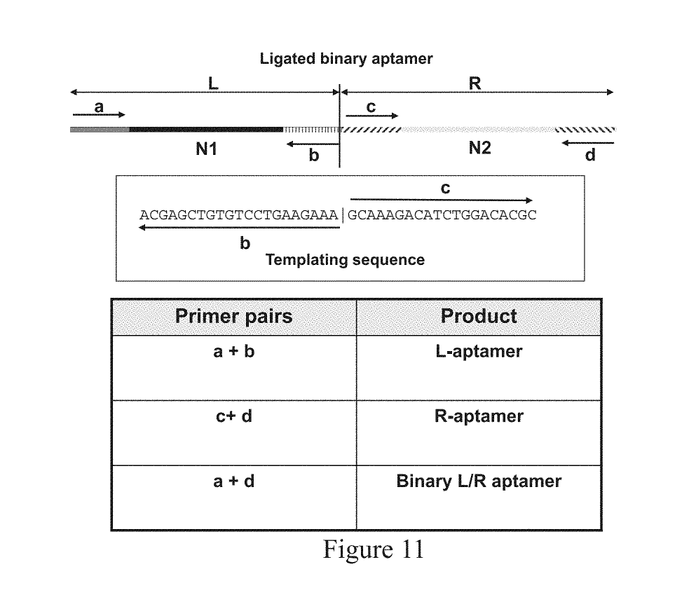

In some embodiments, the clonal analysis procedure can be carried out as described herein. In general, aptamers can be analyzed by cloning and sequencing at any point during the cycling steps, but typically about 9 to 10 cycles are suitable before multiple recurrent clones with high levels of sequence similarity are obtained. After a desired number of cycles, primary eluted Left or Right aptamers can be amplified with appropriate L/R primers (see, FIGS. 3 and 11) to provide a source of duplexes for cloning. Resulting PCR products can be purified to remove excess primers (NucleoSpin kits, Machery-Nagel/Clontech), and then ligated to a vector suitable for direct cloning of fragments produced by Taq DNA polymerase (including, but not limited to, vectors such as pGEM-Teasy, Promega). After an appropriate ligation incubation, competent E. coli cells can be transformed with the products. Mini-preparations of resulting colonies can then be sequenced with primers spanning the aptamer inserts, and analyzed for clones with similar 40-mer tracts.

For instance, selection of specific singlet aptamers on a defined target molecule is depicted in FIG. 4. In brief, the target molecule can be rendered solid-phase after binding to an aptamer population. In some embodiments, the target molecule can be rendered solid-phase by conjugation to N-hydroxylsuccinimide activated magnetic beads. In some embodiments, the target molecules bear one or more biotin moieties, and can be rendered solid-phase by binding to solid-phase streptavidin matrices (such as streptavidin agarose or streptavidin magnetic beads). The non-binding aptamer species can be removed by washing. Bound aptamers can be eluted with 0.05 or 0.1 M sodium hydroxide, precipitated, washed, and then used for re-amplification to obtain enriched single-stranded DNAs for a subsequent round of selection.

Preparation of single-stranded DNA of the correct strand sense from amplified aptamers can allow for the re-iteration of a subsequent round of selection. A preliminary trial amplification can be used to gauge the best concentrations of eluted aptamers to use in bulk PCR preparations to obtain sizable amounts of single-stranded aptamers. In a typical trial amplification (i.e., “range test”), the primary eluted aptamers from each cycle can be diluted at, for example, 1:100, 1:500, and 1:2000, and 1.0 μl of each used in a PCR amplification with Amplitaq Gold (Thermo) with a cycle of 7 minutes at 95° C., 20× (60° C. for about 20 seconds, 72° C. for about 1 minute, and 94° C. for about 40 seconds), 60° C. for about 20 seconds, and 72° C. for about 2 minutes. Products can be analyzed on, for example, a 10% non-denaturing acrylamide gel to determine the concentrations providing the best and purest product yields free from higher-molecular weight forms arising when the starting target concentration is too high. With this information, single strands can be prepared by several different options, including, but not limited to, electrophoresis, denaturation with biotinylated bottom strand, and asymmetric PCR.

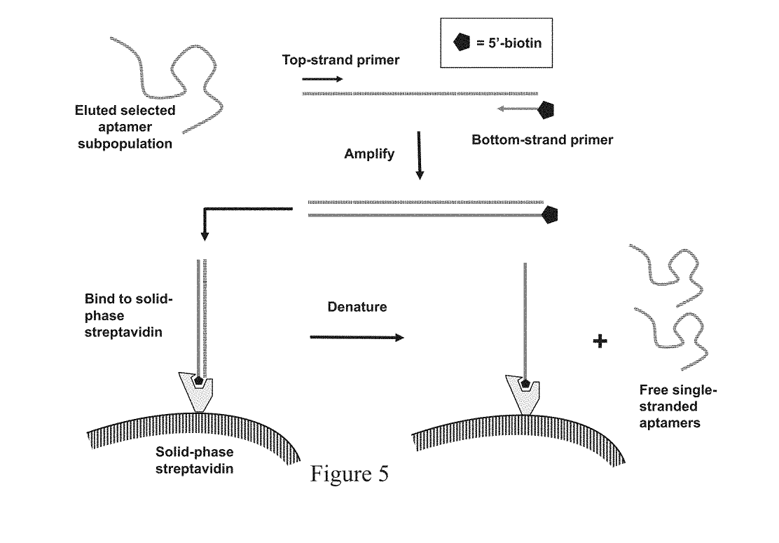

In some embodiments, schematically depicted in FIG. 5, differential strand biotinylation can be used to prepare large amounts of single-stranded aptamer selected subpopulations. Single-stranded aptamer preparations eluted from a solid-phase target can be amplified where the bottom strand (corresponding to the aptamer complement) bears a 5′-biotin. After binding to solid-phase streptavidin, single-stranded aptamers (top strand) can be eluted with alkali (such as, for example, 0.05M or 0.1M NaOH, also with 5 mM EDTA).

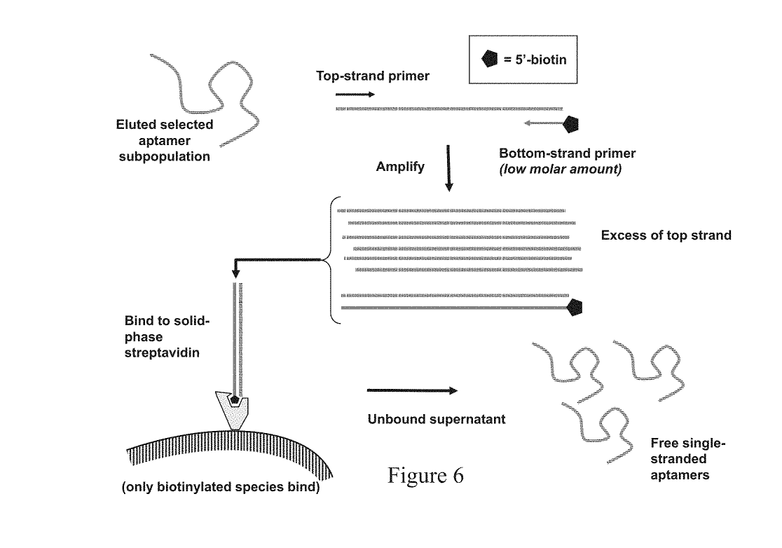

In some embodiments, schematically depicted in FIG. 6, an asymmetric PCR process can be used for generating single-strands from amplified duplex aptamer populations. A large molar excess of top-strand primer can be used, resulting in generation of an excess of single strands corresponding to the desired aptamer subpopulation. Any biotinylated strands can be removed by, for example, binding to solid-phase streptavidin, with the unbound supernatants containing the appropriate single-stranded preparation.

Preparative asymmetric PCR is shown in FIG. 6, and involves an initial amplification of the selected aptamer population where the bottom strand is biotinylated, followed by asymmetric PCR for differential amplification of the top strands. Remaining bottom strands can be removed by binding to SAMBs (as described above, for example).

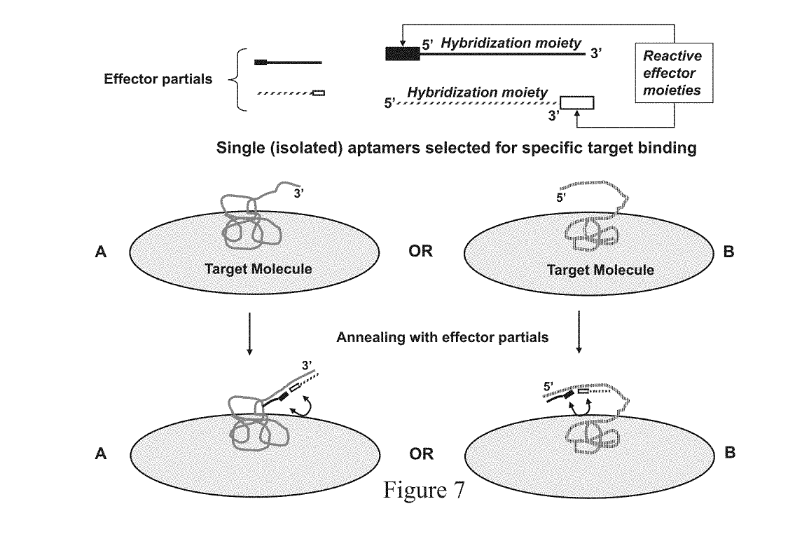

FIG. 7 illustrates a representative singlet aptamer binding a specific target molecule, after which either 3′ or 5′ ends are exposed and accessible to binding by two effector partial molecules (see, FIG. 7A and FIG. 7B, respectively), resulting in a reaction between the bioorthogonally reactive moieties of the latter. Curved arrows denote proximity-induced reaction between different haplomers.

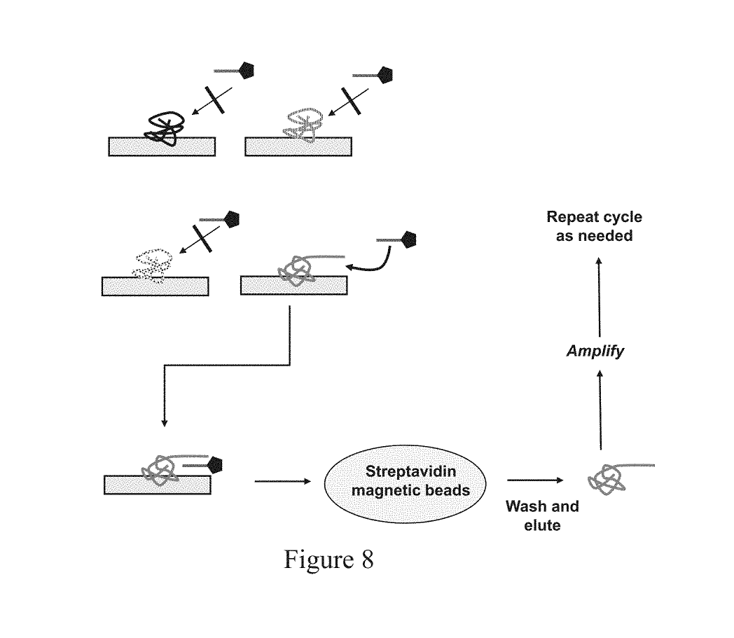

Following selection for binding (see, for example, FIG. 4), singlet aptamers bound to target may not necessarily provide accessible terminal sequences for hybridization, as these may have become incorporated into the folded structures of specific aptamers in the bound state. Singlets with accessible termini can be selected with an additional step, where the singlet aptamers are bound to non-biotinylated targets, and subsequently hybridized with a biotinylated probe complementary to the desired accessible 3′ or 5′ terminus. Since hybridization requires accessibility, appropriate binders can then be selected on a solid-phase streptavidin matrix such as, but not limited to, streptavidin-magnetic beads (see, for example, FIG. 8). Upon elution, singlet aptamers can be amplified and the process repeated if necessary.

In some embodiments, the preparative asymmetric PCR comprises: amplifying the selected aptamer population where the bottom strand corresponding to the aptamer complement is biotinylated, and performing asymmetric PCR for differential amplification of the top strands, whereby a large molar excess of the top-strand primer is used, resulting in generation of an excess of single strands corresponding to the desired aptamer subpopulation.

In some embodiments, biotinylated strands are removed by binding to solid-phase streptavidin, with the unbound supernatants containing the appropriate single-stranded preparation.

The present disclosure also provides methods of selecting an aptamer having an accessible 3′ or 5′ terminal end for hybridization to a haplomer comprising: contacting an aptamer with a corresponding target molecule; contacting the aptamer with a biotinylated probe having a region that is complementary to the 3′ or 5′ terminal end of the aptamer; washing the aptamer-probe complex to remove unbound probe; contacting the aptamer-probe complex with streptavidin magnetic beads; and washing the streptavidin magnetic beads and eluting the aptamer, wherein the aptamer possesses an accessible 3′ or 5′ terminal end for hybridization to a haplomer. This method is shown as one way to select for singlet aptamers presenting accessible sequences after target binding, such that they can be used for subsequent effector partial assembly.

The present disclosure also provides methods of preparing a binary aptamer comprising: contacting a target molecule or target cell with a plurality of aptamers; eluting the bound aptamers; contacting the target molecule or target cell with the population of bound aptamers; contacting the bound aptamers with a ligase and an RNA splint; and removing the splint with RNase H, thereby resulting in a covalently ligated binary aptamer.

A general binary aptamer selection process is described herein. For example, left- and right primary aptamer populations initially selected separately on a specific target (see, FIG. 4) can be co-incubated with the target in equimolar quantities. In a typical procedure, 8 pmol of each of L- and R-aptamers and specific target can be used. After about 2 to 4 hour incubation at room temperature, the target can be bound to a solid-phase matrix as described above (i.e., general binding and elution procedure), and subjected to 4×0.5 ml washes with, for example, PBSM. The solid-phase preparation can be annealed with an excess of splint oligonucleotide spanning the 3′ and 5′ ends of the L- and R-aptamers, respectively (see, FIG. 11). Annealing can be carried out with, for example, incubations of about 5 minutes at about 37° C., and about 30 minutes at about 25° C. Preparations can be washed twice with, for example, ×1 ligase buffer with 1 mM ATP (New England Biolabs), and resuspended in about 50 μl of the same ligase buffer. Ligase can be added, and the preparations incubated for about 1.5 to about 4 hours at room temperature. Controls can be used where the splint and ligase, or both, are omitted.

In some embodiments, the ligase is T4 DNA ligase, T3 DNA ligase or Chlorella DNA ligase (SplintR® ligase; New England Biolabs, with corresponding buffers).

In some embodiments, the ligase is T4 DNA ligase or Chlorella DNA ligase.

In some embodiments, the aptamers can be selected to bind to a cancer cell, and wherein aptamers that bind to normal cells can be subtracted.

Ligation of singlet aptamers co-binding on a common target molecule in spatial proximity results in a continuous fusion between Left and Right aptamers, termed a binary aptamer (see, for example, FIG. 11). From any specific binary aptamer or population of binary aptamers, the entire binary sequence can be amplified with a single pair of primers spanning the joined sequence. If desired, from any specific binary aptamer or population of binary aptamers, component Left and Right aptamers can also be amplified (see, FIG. 11).

Binary aptamers offer the advantages of enhanced specificity and affinity, and afford a templating sequence in the interface between the L- and R-aptamer segments. This sequence has a dual role both for templating desired assembly reactions, and also as primer sites for the L-aptamer reverse primer, and R-aptamer forward primer (see, FIG. 11).

FIG. 11, for example, shows a general binary aptamer structure (L, R=Left and Right aptamer sequences, respectively (derived from original corresponding libraries; see FIG. 1); a, d, c, d=primer sites/primers). The inter-aptamer sequencing used for templating is also shown, with the specific primer sequences for reverse L-aptamer (“b”, antisense in this diagram) and forward R-aptamer (“c”, sense in this diagram). The vertical line in this sequence indicates the ligation junction between L- and R-aptamers.

Generally, binary aptamers conform to the general pattern: (L-forward primer)-(L-random region)-(L-reverse primer/half-splint region)-(R-forward primer/half-splint region)-(R-random region)-(R-reverse primer). The joined (L-reverse primer/half-splint)-(R-forward primer/half-splint) segment constitutes the site whereby a splint molecule enables L- and R-ligation, and also serves as single-stranded accessible template for template reactions.

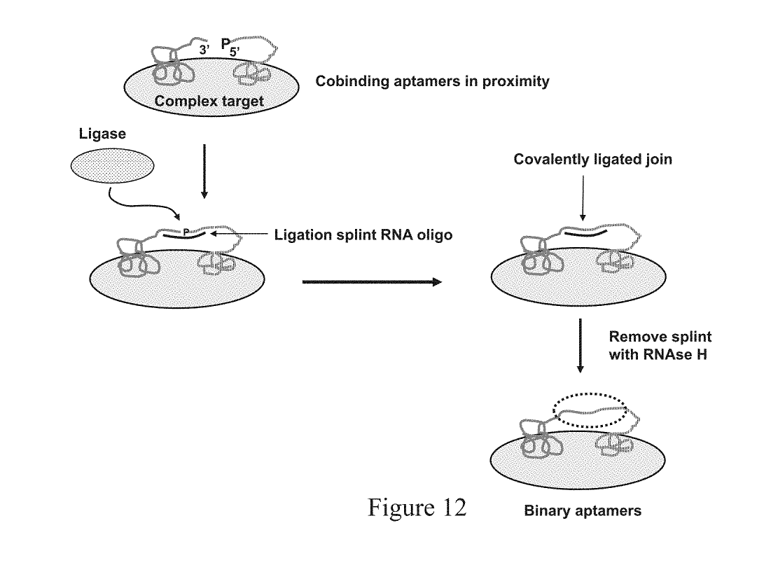

The joining of each Left and Right aptamer into a binary form can be effected by, for example, means of an RNA splint oligonucleotide complementary to the 3′ end of the Left aptamer and 5′ end of the Right aptamer (see, FIG. 12). The ligation of the aptamer ends upon this splint can be effected by T4 DNA ligase, or more efficiently by Chlorella DNA ligase (New England Biolabs), which is highly effective in the ligation of DNA ends by RNA splints. Specific binary pairs can be identified and characterized by amplifying the proximal binary units as a single contiguous sequence (see, FIG. 11, primers a+d). After ligation is complete, the RNA splint can be removed by treatment with RNAse H (which is active only on RNA:DNA hybrids), to expose the joined template region from the Left and Right aptamers for subsequent hybridization with haplomers.

Selection of binary aptamers by target co-binding and splint ligation also simultaneously ensures that the template region is accessible for hybridization purposes. Pairs of aptamers in spatial proximity whose 3′ and 5′ ends are inaccessible (as a consequence of their specific target binding) will fail to hybridize with the splint and allow subsequent ligation and amplification as binary entities (see, FIG. 12).

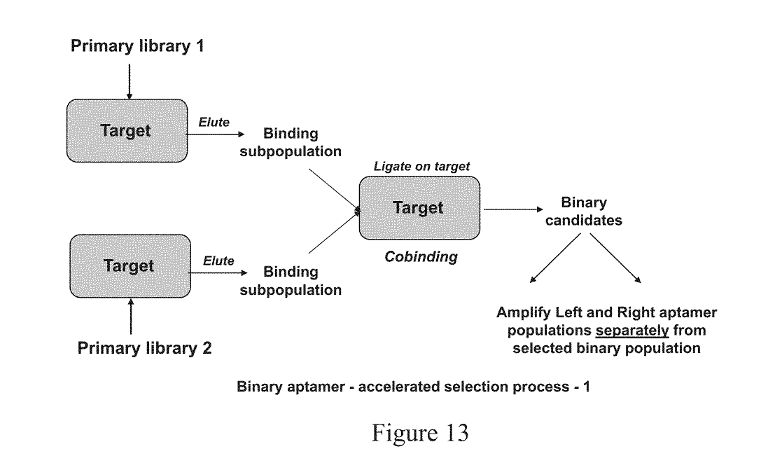

Alternate aptamer selection processes are also disclosed herein. For example, Left and Right aptamer libraries can be initially selected separately on a desired target molecule, and the binding subpopulations eluted (see, FIG. 13). These can then be subjected to co-binding selection for enrichment in proximal binary aptamers, and from the eluted binary populations component Left- and Right-aptamer populations can be amplified (see, FIG. 13). Both of these selected populations can be subjected to recombinatorial DNA shuffling (Stemmer, Nature, 1994, 370, 389-391) to enhance molecular diversity.

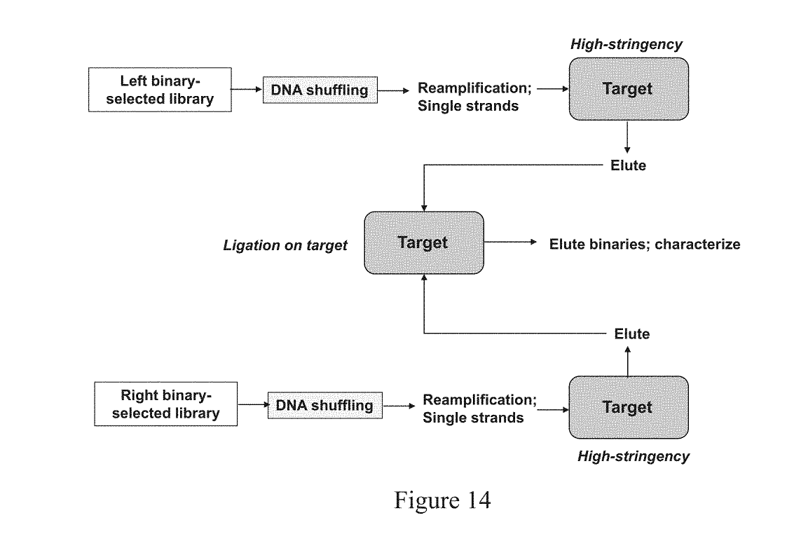

The DNA shuffling step (see, molecular breeding, Stemmer 1994) is designed to promote cross-over priming between different aptamer strands, and is effected by limited DNase I digestion of each selected Left and Right aptamer subpopulations, followed by a reassembly cycle, and then re-amplification with the original primers (see, FIG. 14). Products of aptamer DNA shuffling can be selected once more on solid-phase target at high stringency, followed in turn by co-binding ligation (see, FIG. 13), elution, and amplification. Products of this process can be characterized by sequencing and tests for binding affinity.

The manipulations of both singlet and binary aptamers for templated assembly purposes are described as above. Binary aptamer applications can be divided into two categories. In the first category, following their identification from proximally-binding singlets, binaries can be ligated together in solution (in absence of a target molecule) and then deployed for functional purposes. In the second category, binaries are assembled directly on the target molecule, whether through convenience or necessity.

All aptamers generated for adaptive templating purposes can have their binding affinities measured (as indicated by their K_{d} value). Such affinity measurements can be conducted by various methods, including, but not limited to, BiaCore instrumentation, equilibrium dialysis, gel shift assays, filter-binding assays, and quantitative PCR combined with a separation process for bound and unbound material (see, Jing et al., Anal. Chim Acta, 2011, 686, 9-18).

During any application of templated assembly of haplomers, the haplomer hybridization to a desired template can be specific. Non-specific hybridization can be minimized by selecting target molecules that are unique to the cell type of interest. In cases where only a point mutation distinguishes the target molecule, the risk of off-target molecule hybridization is significant. The use of aptamers to provide templates for haplomers provides a unique opportunity to completely eliminate non-target molecule hybridization.

DNA analogs with L-ribose (L-DNA) instead of D-ribose require homochiral complementary nucleic acid strands for duplexes to form. Thus, a template composed of L-DNA cannot hybridize with any natural nucleic acids, which all possess D-ribose. L-DNA template tags can be appended to aptamers, for the purpose of templating effector partial moieties whose hybridization portions also are comprised of L-DNA. L-DNA haplomers are also advantageous in that their hybridization portions are highly resistant to all nucleases. Single strands of L-DNA are not to be confused with left-handed DNA duplexes (Z-DNA).

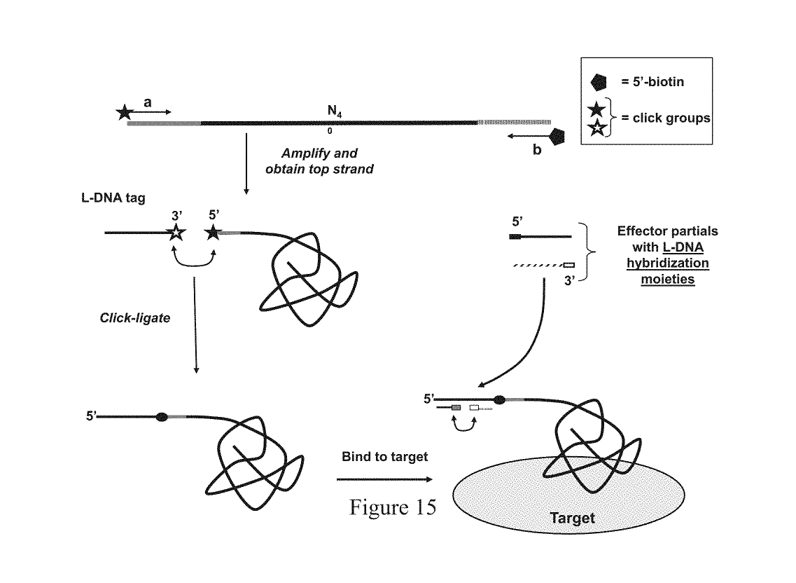

In some embodiments of the methods for aptamer-displayed bioorthogonal hybridization, the accessible 5′ end of a pre-defined singlet aptamer is derivatized with an L-DNA sequence tag, via mutually reactive click chemistry. A 5′ click group is introduced into the aptamer via amplification with a suitable modified top-strand primer (see, FIG. 15), where the bottom-strand primer bears a 5′ biotin to facilitate generation of top (aptameric) single strands (see, FIG. 5 or FIG. 6). After chemical ligation with an excess of a desired L-DNA bearing a 3′ click group (mutually reactive with the 5′ click group carried by the aptamer), the aptamer carries a 5′ tag corresponding to the desired L-DNA sequence. Upon target binding, the L-DNA tag can act as a template for haplomers, but only if these haplomers likewise carry complementary L-DNA hybridization portions.

In some embodiments of the methods for aptamer-displayed bioorthogonal hybridization, the accessible 3′ end of a singlet aptamer is derivatized with an L-DNA sequence tag, via mutually reactive click chemistry. In this case the 3′ end of a pre-defined aptamer is enzymatically ligated via RNA ligase I with a short oligonucleotide sequence (dT₆₋₈) bearing a 5′ phosphate and a 3′ click group. The aptamer 5′ end in this instance bears a 5′ hydroxyl group. Following this, chemical ligation can be carried out with an excess of a desired L-DNA bearing a 5′ click group (mutually reactive with the 3′ click group carried by the aptamer). The resulting aptamer product carries a 3′ tag corresponding to the desired L-DNA sequence. Upon target binding, the L-DNA tag can act as a template for haplomers, but only if these haplomers likewise carry complementary L-DNA hybridization portions.

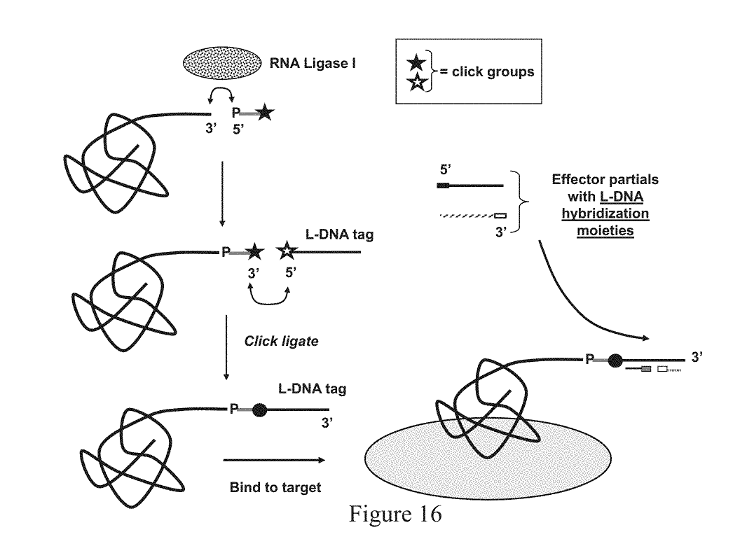

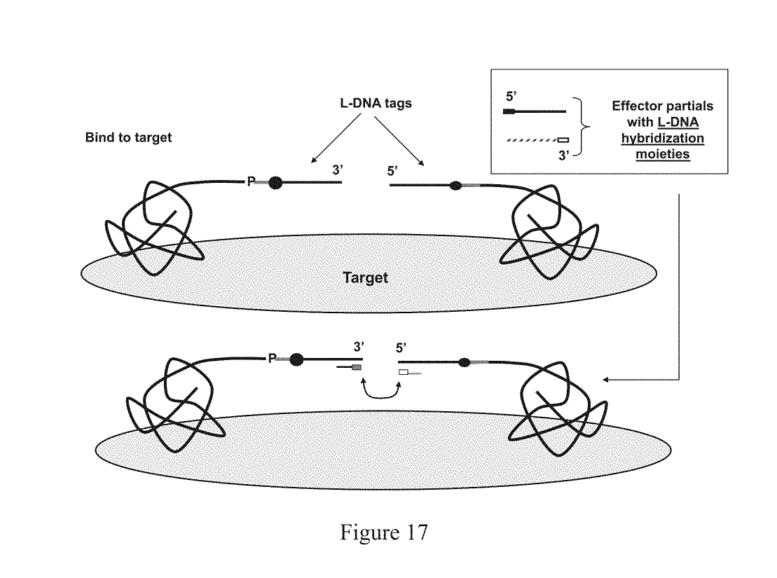

In some embodiments of the methods for aptamer-displayed bioorthogonal hybridization, dual aptamers binding in spatial proximity to a designated target are used to display L-DNA templates. This approach uses appropriate Left and Right aptamers (pre-defined as binding proximally to the desired target molecule by co-binding ligation; see, FIG. 12) bearing 5′ and 3′ L-DNA tags, respectively, as detailed in FIG. 15 and FIG. 16, respectively. In this instance, the haplomers with L-DNA hybridization portions are used, but haplomers are not directed solely to the 5′ end of a single aptamer (as in FIG. 15) or the 3′ end of a single aptamer (as in FIG. 16). Instead, the haplomers are directed to the termini of each of the aptamers of the dual aptamer pair, such that bioorthogonal reactivity is promoted via spatial proximity of the dual aptamer binding on a common target molecule (see, FIG. 17).

Referring to the drawings in particular, FIG. 12 shows the formation of a binary aptamer from a pair of aptamers co-binding proximally close target sites on a complex molecule. The ligation of the aptamer ends upon this splint can be effected by T4 DNA ligase, or more efficiently by Chlorella DNA ligase, which is highly effective in the ligation of DNA ends by RNA splints (New England Biolabs). Following ligation, the splint can be removed with RNase H. The dotted oval indicates accessible template provided by the binary aptamers after RNA splint removal.

In particular, FIG. 15 shows an unnatural L-DNA tag appended onto the 5′ end of a singlet aptamer. A pre-defined aptamer is re-amplified, where the top strand primer bears a 5′ click group, and the bottom strand primer bears a 5′ biotin. After amplification, single strands corresponding to the original aptamer sequence can be prepared (as for FIG. 5 or FIG. 6). The resulting aptamer can then be reacted with an excess of an L-DNA tag of defined sequence, bearing a 3′-click group, orthogonally reactive with the aptameric click group. After binding its target molecule, the aptamer displays the appended L-DNA sequence as a 5′ template, which haplomers bearing complementary L-DNA hybridization portions can recognize. The curved arrows denote a proximity-induced reaction between different haplomers.

In particular, FIG. 16 shows an unnatural L-DNA tag appended onto the 3′ end of a singlet aptamer. A pre-defined aptamer with an accessible 3′ end is ligated with a short single stranded oligonucleotide (such as dT₈) bearing a 5′ phosphate and a 3′ click group, by means of RNA ligase I. The resulting aptamer can then be reacted with an excess of an L-DNA tag of defined sequence, bearing a 5′-click group, orthogonally reactive with the aptameric click group. After binding its target molecule, the aptamer displayed the appended L-DNA sequence as a 3′ template, which haplomers bearing complementary L-DNA hybridization portions can recognize. The curved arrows denote a proximity-induced reaction between different haplomers.

In particular, FIG. 17 shows unnatural L-DNA tags appended onto the 3′ and 5′ ends of dual aptamers, for directing spatial proximity of haplomers by bioorthogonal hybridization. The L-DNA tags at the 3′ and 5′ ends of aptamers proximally binding the same target molecule can be appended separately by the methods of FIG. 15 and FIG. 16.

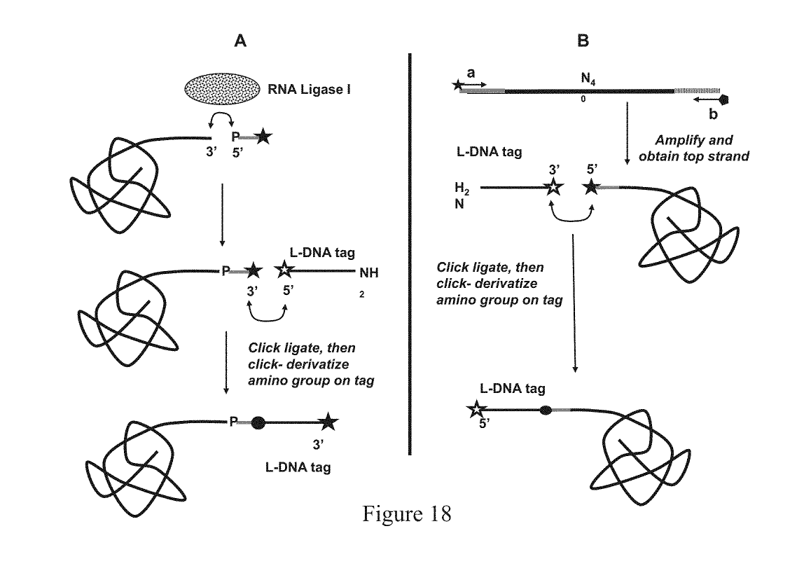

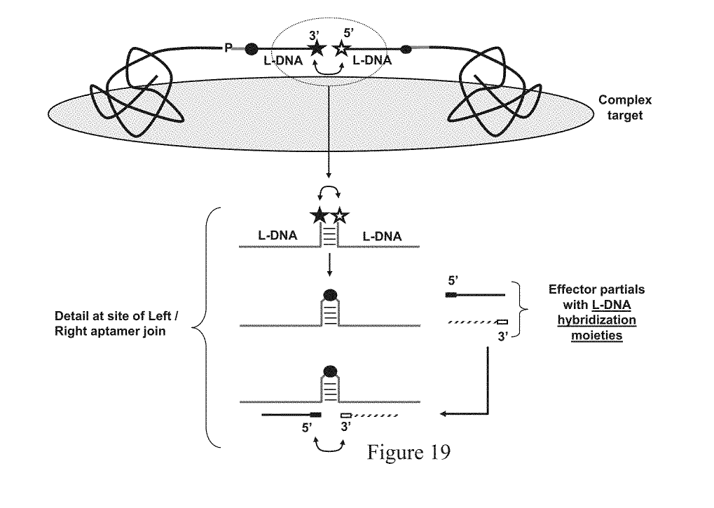

In some embodiments of the methods for aptamer-displayed bioorthogonal hybridization, binary aptamers are used to display L-DNA templates. To achieve this, a double-derivatization process is used. Initially, singlet aptamers comprising the Left and Right segments of a binary pre-selected for proximity by co-binding (see, FIG. 12) are derivatized with L-DNA tags in the same manner as for FIG. 16 and FIG. 15, respectively. In this instance, the L-DNA tags also have amino groups appended to their 3′ and 5′ ends, respectively (see, FIG. 18; panels A and B). Following the initial chemical ligations of each L-DNA tag sequence, the amino groups can be derivatized with appropriate click groups, via N-hydroxylsuccinimide chemistry. These reactions can be performed, since once the previous click groups have reacted, the products are inert towards a second derivitization. The fully derivatized L-DNA tagged aptamers can be in turn chemically ligated together by co-binding to the target molecule of interest. In this instance, the interaction between each L-DNA tag is facilitated by a short (i.e., 4-6 base) mutually complementary terminal sequence (see, FIG. 19). This forms a short stem loop, which in turn facilitates the subsequent reaction of hybridizing L-DNA haplomers (see, FIG. 19), by enhancing spatial proximity, as previously shown with oligonucleotides bearing click-reactive groups.

In particular, FIG. 18 shows equipping binary aptamers with bridging unnatural L-DNA sequences, for directing spatial proximity of haplomers by bioorthogonal hybridization. Panel A shows the preparation of Left aptamers with derivatized L-DNA tags. The initial linkage of the L-DNA tag is as for FIG. 16, except that the L-DNA bears a 3′-amino group for a secondary derivatization with a click group. Panel B shows the preparation of Right aptamers with derivatized L-DNA tags. The initial linkage of the L-DNA tag is as for FIG. 15, except that the L-DNA bears a 5′-amino group for a secondary derivatization with a click group. In both cases, the secondary derivatizations can be performed since once the previous click groups have reacted, the products are inert towards a second derivitization.

In particular, FIG. 19 shows equipping binary aptamers with bridging unnatural L-DNA sequences, for directing spatial proximity of haplomers by bioorthogonal hybridization. Chemical ligation on the target molecule of Left and Right derivatized aptamers bearing L-DNA sequences, and subsequent hybridization with haplomers is shown. Each Left- and Right L-DNA segment is designed to a have a short (i.e., 4-6 base) mutually complementary sequence to facilitate both local interaction and subsequent haplomers spatial proximities.

In some embodiments, the click-reactive groups can be, but are not limited to, azide and strained cyclooctyne groups, or tetrazine derivatives and trans-cyclooctene groups. For 5′ template modification, top-strand primers can be initially synthesized with a 5′ amino group, which can be subsequently converted into the appropriate click group through reaction with a click group-N-hydroxylsuccinimide moiety.

Many cases of ligand-induced allosteric structural changes have been documented with both RNA and DNA aptamers. Such effects have been usefully exploited for the generation of specific aptameric functionalities, such as aptabeacons and aptasensors. In this instance, selection for allosteric effects can be performed such that aptamer-derived template is only exposed for effector partial moiety hybridization after binding to the target molecule. Allosteric aptamers of these types add additional power to the utility of aptamers as display vehicles for template assembly. Specifically, an aptamer system where the accessible template is only exposed after target molecule binding promises to reduce non-specific haplomer interactions. In other words, in an environment where the aptamer encounters no specific target, no template is accessible for templated assembly either.

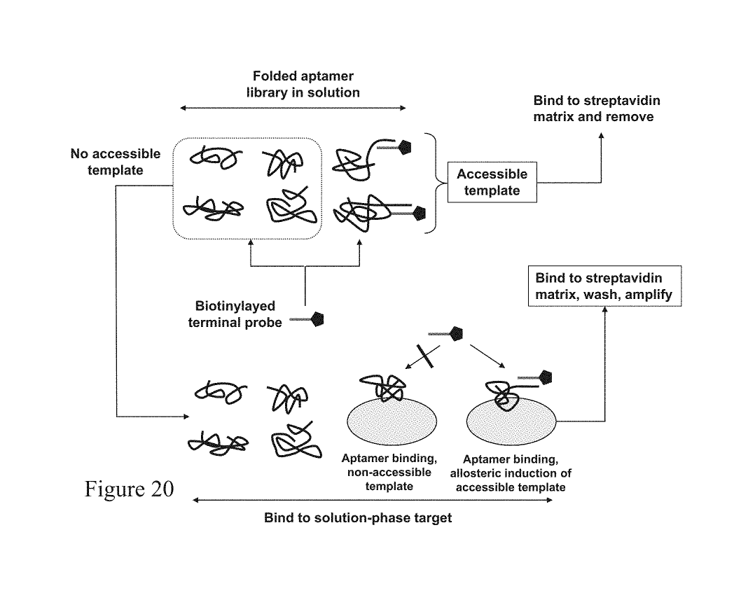

In some embodiments for the methods for selection of allosteric aptamers for haplomer applications, a singlet aptamer is selected where the terminal template sequence for template assembly is only exposed and accessible after aptamer binding to the specific target molecule. This process is depicted schematically in FIG. 20, and involves a cycle of negative and positive selections. The first step involves partitioning an unselected aptamer library into those members with accessible termini in solution, and those members whose termini are not accessible to hybridization, as defined with a biotinylated probe sequence. Solution-accessible members of the library can be removed by binding of the annealed probe to, for example, a solid-phase streptavidin matrix. This process accordingly negatively selects for folded aptamers in solution whose template sequences are not accessible to an added probe molecule. Within this population, a second positive selection (again by means of, for example, a biotinylated probe sequence) can be made for members which generate accessible template sequences as a consequence of target binding (see, FIG. 20). This positive selection is analogous to the selection process for singlet aptamers with accessible targets, as depicted in FIG. 8. When resulting selected aptamers are amplified and the appropriate single-stranded preparations made, the process can be repeated as a cycle. When the heterogeneity of the selected population after N cycles is highly reduced, the resulting population can be cloned and individual aptamers screened. Candidate aptamers can conform to the original selection criteria as being refractory to template-based interactions in free solution, but amenable to such interactions in the presence of specific template. Allosterically-induced accessible template can also permit the templated assembly of haplomers.

In particular, FIG. 20 shows the selection process for aptamer allostery, where target molecule binding induces the exposure and/or accessibility of the template sequence. Aptamers whose templating sequences are accessible in solution (before presence of a target molecule) can be removed by initial hybridization to, for example, an appropriate biotinylated probe sequence, and immobilization on, for example, a solid-phase streptavidin matrix. The supernatant fraction can be incubated with target molecule in solution. Aptamers which bind to the target molecule and undergo an allosteric change which renders the template sequence accessible are selectable by, for example, biotinylated probe binding. Those aptamers whose template sequences remain masked or inaccessible are not. Therefore, sequestration of the former on, for example, a solid-phase streptavidin matrix allows their selective amplification. The eluted aptamer preparations obtained in this manner can be then subjected to a repeat of the whole cycle. Cycling can be performed until analyses of the resulting populations show highly reduced homogeneity, after which analysis of specific cloned aptamers can be carried out.

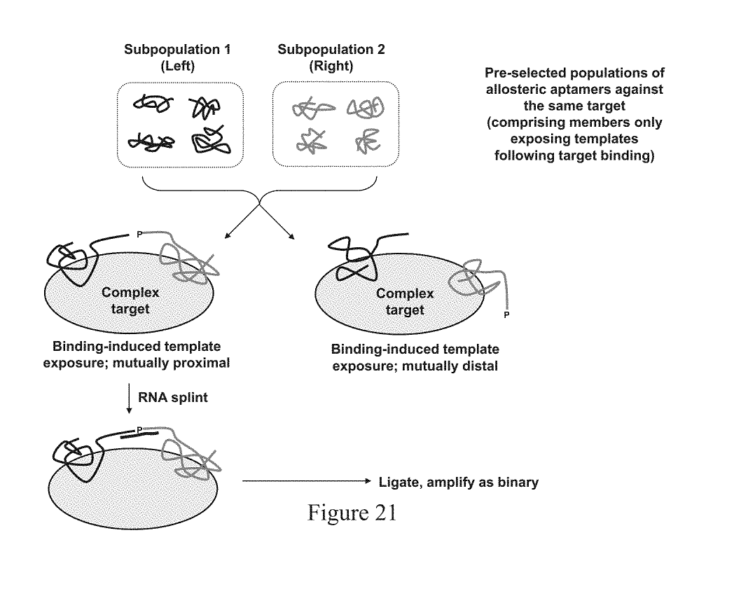

In some embodiments of the methods for selection of allosteric aptamers for effector partial moiety applications, binary aptamers are selected where the 3′ and 5′ ends of each singlet component comprising the binary form are only exposed in accessible proximity following target molecule binding. Both Left and Right aptamers directed towards a target of interest can be initially derived in the same manner as the methods described herein, where both aptamers exhibit allosterically exposable template sequences only following interaction with a target molecule. Populations of such target-directed aptamers can be subjected to the co-binding process and splint-directed in situ ligation. Specific binary pairs can be identified and characterized by amplifying the proximal binary units as a single contiguous sequence (see, FIG. 11, primers a+d). When specific pairs are identified, splint removal can be effected by using RNase H (as in FIG. 12), after which the junctional template sequence is available for haplomer templated assembly. This allosteric binary process is outlined in FIG. 21.

In particular, FIG. 21 shows application of aptamer allostery towards the in situ generation of joined binaries. The linking templating sequences between each Left and Right component of a binary aptamer pair are only available following target molecule binding and allosteric exposure of terminal templates in spatial proximity Such pairs can be identified by co-binding on the original target, RNA splint-mediated ligation, and amplification. Once a specific binary aptamer pair have been identified, they can be used for haplomer templating in the same manner as detailed in, for example, FIG. 12.

The present disclosure also provides methods of delivering at least one aptamer to a pathogenic cell. In some embodiments, the method comprises: administering a therapeutically effective amount of any one or more aptamers described herein to the pathogenic cell. In some embodiments, at least one active effector structure in the pathogenic cell is produced. In some embodiments, the aptamer is administered separately from one or both haplomers. In some embodiments, at least one of programmed cell death of the pathogenic cell, apoptosis of the pathogenic cell, non-specific or programmed necrosis of the pathogenic cell, lysis of the pathogenic cell, and growth inhibition of the pathogenic cell is produced. In some embodiments, the pathogenic cell is selected from the group consisting of a virus infected cell, a tumor cell, a cell infected with a microbe, and a cell that produces a disease-inducing or disease modulating molecule that may cause inflammation, allergy or autoimmune pathology.

Referring to the drawings, FIG. 1 shows a representative method for protein or other macromolecular targets. Unlike nucleic acid targeting by digitally-based hybridization rules, this form of templating is shape-based, and thus can be considered “analog” in nature. The curved arrows denote proximity-induced reaction between different haplomers.

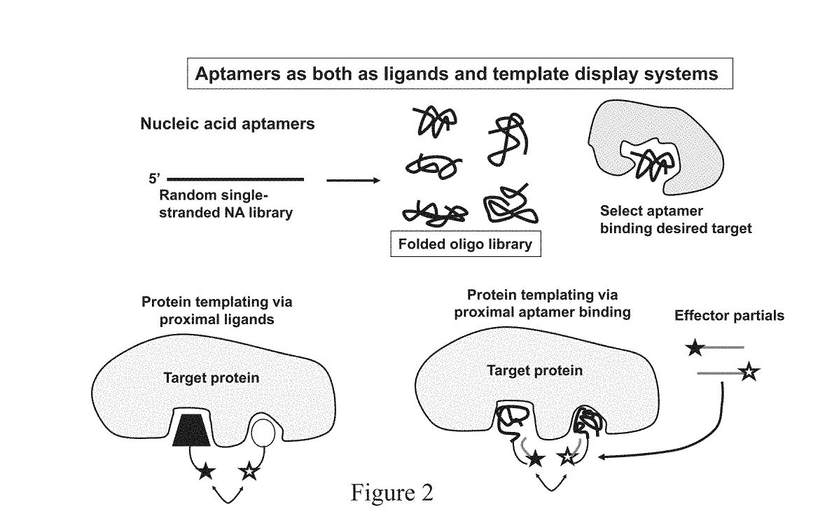

In particular, FIG. 2 shows a representative deployment of aptamers for non-nucleic acid macromolecular templating. In some embodiments, regions of aptamer sequences are used for hybridization-based templating of haplomers bearing bioorthogonal reactive groups. The curved arrows denote proximity-induced reaction between different haplomers.

In some embodiments, the pathogenic cell is a virus infected cell and the method produces at least one of programmed cell death of the virus infected cell, apoptosis of the virus infected cell, non-specific or programmed necrosis of the virus infected cell, lysis of the virus infected cell, inhibition of viral infection, and inhibition of viral replication. In some embodiments, the pathogenic cell is a tumor cell and the method produces at least one of programmed cell death of the tumor cell, apoptosis of the tumor cell, non-specific or programmed necrosis of the tumor cell, lysis of the tumor cell, inhibition of the tumor cell growth, inhibition of oncogene expression in the tumor cell, and modification of gene expression in the tumor cell. In some embodiments, the pathogenic cell is a microbe-infected cell and the method produces at least one of programmed cell death of the microbe-infected cell, apoptosis of the microbe-infected cell, non-specific or programmed necrosis of the microbe-infected cell, lysis of the microbe-infected cell, inhibition of microbial infection, and inhibition of microbe replication.

In some embodiments, tumor cells are targeted by aptamers to allow selective cell killing by template assembly. In some embodiments, specific proteins or post-translationally modified proteins, protein complexes, carbohydrates, lipids, phospholipids, glycolipids, nucleic acids, and ribonucleoproteins can be targeted for aptamer binding and template presentation. The specific targets can be altered in some manner from the normal form such that they are restricted to cell lineage-specific, or any tumor cells, or altered in their normal cellular localization. Designated target molecules may be localized to cell surfaces, or found intracellularly, either within the cytoplasm or nucleus.

In some embodiments, where mutated tumor proteins have altered conformations, they provide useful targets for aptamer-mediated template presentation for the purposes of template assembly. Such conformational changes include, but are not limited to, misfolding and exposure of normally internalized residues, the induction of prion-like domains, and altered protein-protein interactions.

In some embodiments, tumor-specific protein target molecules are desired, and are potential targets for aptamer-based templated assembly. These include, but are not limited to, mutated oncogenes, growth factors, cell cycle regulators, and transcription factors.

In some embodiments, non-protein molecular tumor markers are desired, and are potential targets for aptamer-based template assembly. As a non-limiting example, phospholipids (including, but not limited to, phosphatidylserine and phosphatidylethanolamine) can be abnormally expressed on the exterior of tumor cells and tumor-associated vasculature in an “inside-out” manner.

In some embodiments, the target molecules within pathogenic cells may not necessarily be present initially, but become expressed as a consequence of specific prior or concurrent drug treatments. As one non-limiting example of tumor-specific marker expression induced by drugs, demethylating agents can induce endogenous retroviral sequences preferentially in colorectal cancer cells (Roulois et al., Cell, 2015, 162, 961-973). As another non-limiting example of this effect, abnormal surface phospholipid expression in tumors may in some cases be selectively enhanced by conventional cytotoxic drug treatments.

In some embodiments, abnormal clustering of surface molecules occurring during tumor cell development can be exploited as a target for aptamer-based template assembly. As a non-limiting example, it is known that both the composition of cell surface glycans and glycoproteins is markedly altered for certain tumor cells (Paszek et al., Nature, 2014, 511, 319-325), with resulting increased surface clustering of other molecules. As a result, important signaling proteins such as integrins attain increased spatial proximity on such tumor cell surfaces in comparison to matched normal tissue cells. Consequently, in some embodiments, aptamers can be developed against suitable surface-expressed integrins.

In some embodiments, various other pathogenic cells are targeted. These include, but are not limited to, pathogenic immune cells or immune cells whose removal is beneficial to a human or animal. In such cases, specific molecular targets include, but are not limited to, idiotypic domains of antibody or T cell receptors of clonal B or T cells respectively, cell lineage-specific surface markers, and cell lineage-specific cytokines.

In some embodiments, virally-infected cells are targeted. Viral-specific targets can be intracellular viral transcripts or host transcripts induced into abnormal expression patterns as a consequence of viral infection, or surface structures also manifested as a result of viral replication. Non-limiting examples of the latter include abnormal surface expression of phospholipids such as phosphatidylserine.

In some embodiments, tumor-specific target molecules for aptamer-based template assembly can be uncharacterized, especially as individual tumors that undergo progressive evolutionary changes in vivo, associated with increasing tumor heterogeneity. Here, novel aptameric targets can be isolated by physical subtractive approaches, by means of matched normal cells of equivalent lineages. Initially, a specific tumor cell type of interest is used, and also a matched normal control cell type for subtractive purposes. In lieu of the latter, and particularly when multiple biopsy samples have been taken progressively over time, tumor samples at an earlier stage of evolutionary progression can be used as the “subtractor” material.

In some embodiments, tumor and cognate normal cell samples can be of various descriptions. These include, but are not limited to, whole cells (for selection of cell-surface targets), whole cell cytoplasmic lysates (selecting for all intracellular targets, including protein, RNA, and ribonucleoproteins), or whole RNA.

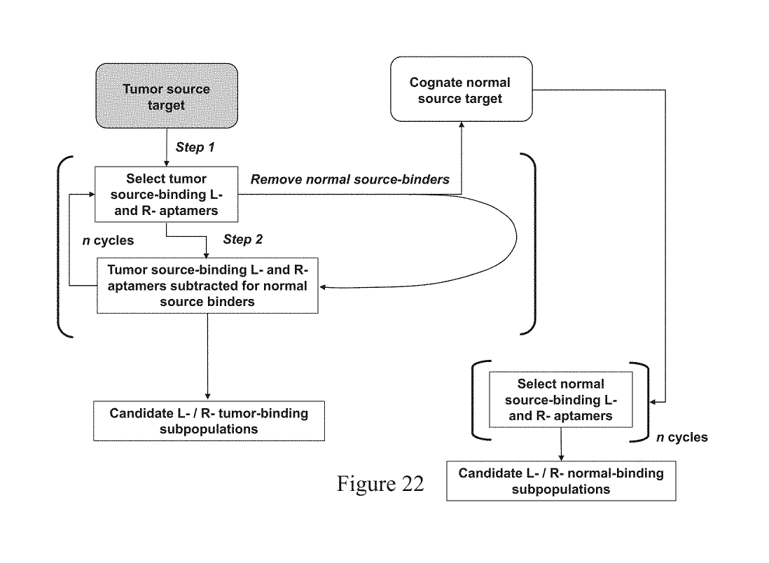

In some embodiments, an initial round of Left- and Right-aptamer selection for binders is performed using the tumor-derived source material of interest (see, FIG. 22). If the source material is whole cells, unbound material can be removed during binding selection by low-speed centrifugation and washing. If the source material is whole-cell cytoplasmic lysate or whole cell RNA, unbound material can be removed by, for example, differential PEG precipitation. This step can be followed by a subtractive removal of aptamers binding to material from cognate normal sources, where the separation of bound and unbound is the same as in the initial step. These steps can be repeated through a series of cycles as appropriate (10 such cycles are usually sufficient).

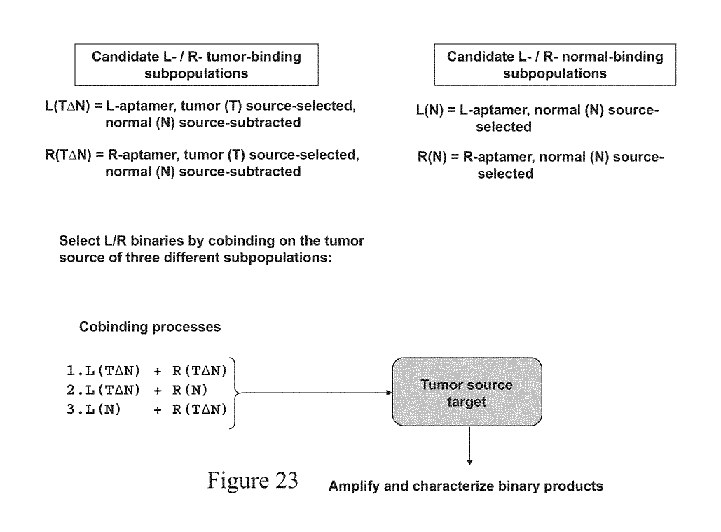

In particular, FIG. 22 shows subtraction between aptamers binding targets from a tumor cell source and those binding a matched cognate normal cell. The source material can be whole cells (selecting for cell-surface targets), whole cell cytoplasmic lysates (selecting for all intracellular targets, including protein, RNA, and ribonucleoproteins), or whole RNA. L- and R-aptamer libraries can be initially used to select subpopulations which bind to tumor sources, and which escape removal by binding to corresponding normal source targets, in order to enrich for aptamers exclusively binding tumor-related molecules. This binding and subtraction process can be repeated for a suitable number of cycles (n, as shown). L- and R-aptamer libraries directly binding such normal counterparts to the tumors can also be directly selected for the next stage of the process, using the same number of cycles (see, FIG. 23).

In a variation of such methods, the normal source material of interest (corresponding to the tumor source material) can also be used to select for binding Left- and Right-aptamer populations directly, where the separation of bound and unbound is the same as described above. The resulting subpopulations of Left- and Right-aptamers binding normal target molecules can be used for the subsequent selective purposes.

Following the steps outlined above after appropriate cycling, the selected subpopulations of Left- and Right-aptamers binding tumor sources of interest and subtracted for cognate normal sources can be used for co-binding experiments on the same original tumor sources. These correspond to co-binding process of FIG. 23, where L(TΔN) and R(TΔN) denote Left-aptamers binding tumor sources of interest and subtracted for cognate normal sources, and Right-aptamers binding tumor sources of interest and subtracted for cognate normal sources, respectively. In addition, it can be useful to perform tests with both L(TΔN) and R(TΔN) subpopulations co-bound to a tumor target in conjunction with corresponding Right- and Left-aptamers previously selected for binding to cognate normal targets (R(N) and L(N), respectively). These correspond to co-binding processes depicted in FIG. 23.

In particular, FIG. 23 shows subtraction between aptamers binding targets from a tumor cell source and those binding a matched cognate normal cell. Continuing from FIG. 22, co-binding experiments with L- and R-tumor-binding, normal source-subtracted subpopulations can be performed, (Co-binding process 1) but also with each of these L- and R-populations co-bound with R- and L-subpopulations (respectively) from corresponding normal sources. The use of “half-normal” binaries is for increasing the probability of finding an amplifiable binary product where at least one half has tumor specificity.

A rationale for the subtractive/co-binding processes depicted in FIG. 23 derives from the unknown surface density of novel tumor-specific targets. While a binary composed of both Left- and Right-tumor-restricted epitopes is desired, a singlet epitope that defines a tumor subset is still very valuable. An aptamer recognizing such an epitope in conjunction with a proximal normal epitope retains its ability to recognize the target tumor cell, but also gains the improvements in specificity and affinity associated with binary aptamers.

In some embodiments, the subtraction process involves tumor target cells with and without an in vitro drug treatment. Here, drug-treated whole tumor cells or treated tumor cell extracts can be used to select for L- and R-aptamer binders, and corresponding untreated tumor cells likewise subjected to the same selection processes. For each cycle of selection for the drug-treated cohort, aptamers binding untreated cells or untreated extracts can be removed. Finally, co-binding selection for binary aptamers binding treated tumor targets can be performed, where either or both of the L- and R-components exclusively bind to the treated preparations, analogously as for tumor/normal cells as depicted in FIG. 23.

In some embodiments, non-limiting examples of the drug treatments can be interferon-beta, Hsp90 inhibitors, kinase inhibitors, topoisomerase inhibitors, cytotoxic agents, DNA demethylating agents, or HDAC inhibitors. Within the scope of such methods, any combination of such drug treatments is also included.

In some embodiments, the template assembly process can be effectively exploited for in vitro cellular selection processes, or cellular diagnostics. This is particularly applicable to binary approaches where it is more facile to assemble a binary on a target molecule, as with L-DNA tagged binaries or binary allosteric aptamers. This can be amenable to in vitro applications, particularly for directed identification and selection of rare cellular subsets.

In some embodiments, the cellular types targeted in vitro for identification or selection are comprised of, but not limited to, immune cell subsets, natural stem cell subsets, induced stem cell subsets, endocrine cell subsets, and neural cell subsets.

In some embodiments for diagnostic or research purposes, target cell subpopulations in vitro can be labeled for fluorescence-based cell sorting, by means of, for example, binary fluorescent aptamer binding and effector partial moiety templating. The fluorescent moieties can be carried by either or both aptamers, or through the agency of the reaction between haplomers.

In some embodiments, for diagnostic, therapeutic, or research purposes, target cell subpopulations in vitro can be removed by binary aptamers which deliver a template assembly-mediated killing signal. One example of this method is a negative selection for subpopulations not recognized by the specific binary aptamers used in such circumstances.

In some embodiments, for diagnostic, therapeutic, or research purposes, specific cell subpopulations in vitro can be targeted by binary aptamers which direct the templated assembly-mediated production of a positively selectable marker.

In some embodiments, the selectable marker is comprised of, but not limited to, fluorescent moieties, peptides or other molecular structures for which antibodies are available, or assembled affinity tags for available protein-ligand systems.

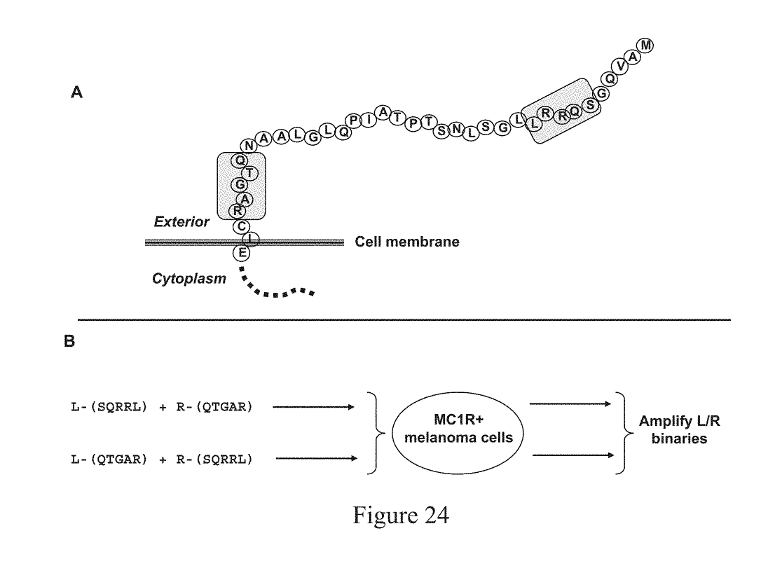

In some embodiments, Left and Right components of binary aptamers are directed towards short contiguous peptides within known target proteins whose structure is available, or whose structure has high conformational flexibility, or whose structure is intrinsically disordered. A non-limiting example of this is the N-terminal extracellular domain of human melanocortin-1 receptor (MC1R), which is comprised of 36 amino acid residues (see, FIG. 24, panel A) and widely expressed on normal melanocytes and melanoma cells. Pentapeptide sequences within this tract can serve as independent aptamer targets, with best candidates bearing a preponderance of charged or hydrophilic residues. The chosen sites, referred to herein as “epitopes” (see, FIG. 24, panel A; SQRRL and QTGAR in order from the N-terminus), also bear one or more arginine residues, which is advantageous for aptamer targeting owing to the positive charge carried by the arginine side-chain at neutral pH (Geiger et al., Nucleic Acids Res. 1996, 6, 1029-1036). While numerous proteins bear either of these pentapeptide sequences, no known proteins (in addition to MC1R) with both sequences exist in current databases.

In particular, FIG. 24 (panels A and B) show N-terminal extracellular domain of the human MC1R protein (panel A). The boxed regions indicate two potential epitopes for aptamer targeting (SQRRL and QTGAR). Panel B shows co-binding experiments with both combinations of L- and R-aptamer subpopulations binding these pentapeptides.

In some embodiments, L- and R-aptamer subpopulations binding separately to SQRRL and QTGAR can be selected by standard procedures (see, FIG. 4). Each combination of L- and R-aptamers against the two pentapeptides can be subjected to the co-binding process on intact melanoma cells previously shown to express MC1R (see, FIG. 12 and FIG. 24, panel B). Specific co-binding of L/R aptamers under such circumstances occurs on MC1R N-termini but not elsewhere. Binary aptamer binding to MC1R allows template assembly for effector partials directed at the melanocytic cell lineage, including melanoma cells.

In a variation of this embodiment, L- and R-aptamers can be selected for D-isomers of the SQRRL and QTGAR sequences. This affords the opportunity to subsequently synthesize L-aptamers (spiegelmers; from the derived sequences of the selected normal aptamers with D-ribose chirality) which recognize the opposite chirality of the original target (normal L-amino acids).

In some embodiments, the accessible short amino acid tracts can be hydrophobic residues specifically exposed on tumor-related proteins through aberrant folding, associated with induction of the Unfolded Protein Response.

In some embodiments, singlet or binary aptamers delivered intracellularly bind to folded RNA sequences such that they act as adaptors to the RNA by means of provision of a targetable template for template assembly. In such an arrangement, the bound aptamers adapt the target RNA for templating purposes, rather than the haplomers using the target RNA sequence itself. This complements conventional template assembly, in circumstances where accessible and efficient sites for direct template assembly in a useful target RNA are absent.

In some embodiments, aptamer-mediated templating of effector partial moieties directs the assembly of peptide epitopes recognized by well-characterized therapeutic antibodies. Such antibodies include, but are not limited to, antibodies recognizing HER-2/neu, EGFR, and VEGF. Where short peptide sequences corresponding to the recognition sites on the target antigens are not available, this embodiment also includes peptide epitope identification with the available antibodies of interest by means of, for example, peptide phage display libraries, as employed in the identification of lymphoma antibody binding specificities.

In some embodiments, an aptamer, whether as a constituent of a binary pair or as a singlet, binds to surface anionic phospholipids, including, but not limited to phosphatidylserine, phosphatidylethanolamine and phosphatidylinositol. In some embodiments, the selection for aptamers binding anionic targets is augmented through the provision of cofactors bearing a positive charge at neutral pH. These include, but are not limited to, small amines such as putrescine, spermine, and spermidine.

In some embodiments, the cell surface target is the human integrin-β1 extracellular domain.

In some embodiments, a ligand for a known surface structure can be covalently tagged with a primer site, and co-binding (proximal) singlet aptamers are sought. After identification of aptamers of interest, the ligand-primer and proximal singlet can be pre-ligated as a binary aptamer with a removable RNA splint, for cell surface templating with haplomers (or click-ligated with L-DNA sequences).

Numerous advantages exist for aptamer-mediated adaptive templating compared to conventional templated assembly. For example, aptamers greatly expand the range of targetable molecules: to proteins, peptides, carbohydrates, particular amphiphilic lipids (e.g., phospholipids), and nucleic acid structures not otherwise targetable by conventional template assembly (such as highly folded RNA secondary structures). Aptamers also allow template assembly to be performed on cell surfaces. Cell-surface templating circumvents many delivery issues, since cell penetration is not required.

Numerous advantages also exist for aptamer-mediated adaptive templating compared to antibody-based alternatives. Conversion of diverse cell surface targets into a common target structure for immune recognition is possible with aptamers. For example, aptamer-mediated recognition of a target cell surface structure allows template assembly of a traceless peptide recognized by a previously developed antibody or a CAR-T system. Also, aptamer-mediated recognition of a target cell surface structure allows templated assembly of a click-ligated peptidomimetic recognized by a previously developed antibody or a CAR-T system. In both of these examples, both the aptamer templating region and the complementary haplomers bearing the reactive half-epitopes are modular, and if L-DNA tags are used, the system can involve bioorthogonal hybridization.

In addition, where target structures are previously known, development of antibody-drug complexes or CAR-T systems is complex and expensive. In contrast, following isolation of a specific recognition aptamer, an adaptive templating system is “ready to use,” and exploits pre-existing template assembly technology.

Where target structures are newly defined, it is much quicker and cheaper to develop a new aptamer that combines target specificity and template assembly template than a corresponding antibody.

Where target structures are unknown, aptamer libraries can be used for subtractive approaches to detect novel surface structures on tumor cells absent from normal cells, or novel structures on drug-treated tumor cells vs. untreated tumor cells. This is impractical or much more difficult in the case of antibody-based technologies.

For all cases where antibodies might be used instead of aptamers, the relatively small sizes of aptamers provides a distinct advantage for tumor cell access in tumor microenvironments. Also, it is much more probable that aptamers can be efficiently transfected within cells (for binding intracellular targets) than large protein molecules such as antibodies.

In order that the subject matter disclosed herein may be more efficiently understood, examples are provided below. It should be understood that these examples are for illustrative purposes only and are not to be construed as limiting the claimed subject matter in any manner Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Demonstration of Co-Binding Selection on a Solid-Phase Target, and Sequence Confirmation of Binary Aptamer Candidates after Co-Binding Selection

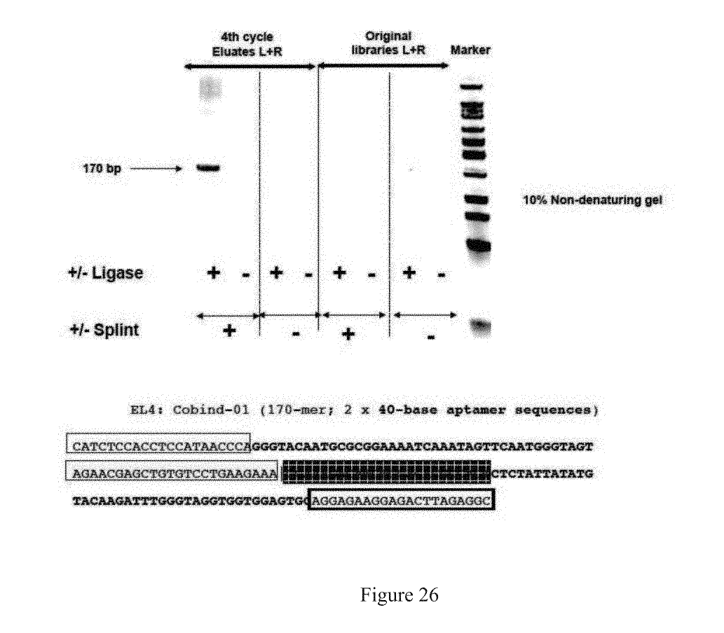

After 4 cycles of separate selection of Left- and Right-aptamer libraries, eluted subpopulations were incubated with the biotinylated immunoglobulin Fab target (against BRD7 protein; ThermoFisher), and then rendered solid-phase by binding to streptavidin-magnetic beads. After 3 washes with 0.5 ml PBSM and 1 wash with 1×T4 DNA ligase buffer (NEB, containing 1 mM ATP), preparations were subdivided into two equal halves and subjected to +/− annealing with the DNA splint (5′-TCCAGATGTCTTTGCTTTCTTCAGG ACACAG (SEQ ID NO:5); 100 μl, 1 pmol/μl), by heating for 5 minutes at 37° C., and then holding for 1 hour at room temperature. Following this, the preparations were again washed 2 times with ligase buffer. Tubes were then split once more into two equal parts, and treated with +/−T4 DNA ligase. Small samples (1 μl) of these reactions were then amplified with primers Trz.F/Trz.R (see, FIG. 25), and tested on 10% non-denaturing acrylamide gels (see, FIG. 26). This showed that a strong product of binary size was observed with the 4th-cycle material, only via the agency of ligase, and only in the presence of the splint. Significantly, a comparable strong product band was not seen from the original (unselected) aptamer Left- and Right-libraries (see, FIG. 26). This demonstrates that the cycles of binding, washing, and amplification had significantly enriched for Fab-selective binders over the original unselected populations. Sequencing revealed that the amplified product from the co-binding test for the 4th cycle material showed perfect fusion of the Left- and Right-aptamer components, as joined via the splint oligonucleotide (see, FIG. 26).

In particular, FIG. 26 demonstrates successful co-binding after 4 cycles of Fab selection, and sequence analysis of a co-binding experiment binary aptamer product. Cobind-01, arbitrary example of cloned product from EL4 Left- and Right-aptamer populations subjected to co-binding process (see, FIG. 12). Bold sequences are 40-mer tracts deriving from randomized sequence in the original aptamer libraries. Boxed sequences are primer sites. No fill is primer Trz.F. Light gray fill is primer AptInt.R (antisense in this orientation). Speckled fill is primer AptInt.F. No fill, dark lines is primer Trz.R (antisense in this orientation). This specific sequence example can be compared with the general structure of binary aptamers, as depicted in FIG. 11.

### Example 2: Singlet Aptamer Analysis and Binary Aptamer Generation (Co-Binding Process) after 10 Cycles of Selection on Fab Fragments

After 10 cycles of separate selection for singlet Left- and Right-aptamer subpopulations on biotinylated Fab target (see, FIG. 4), the resulting subpopulations were cloned and sequenced. Here (in contrast to results from the 4th cycle), multiple recurrences of a specific aptamer sequence were observed. From 14 sequenced specific singlets (7 each from Left- and Right-clone populations), 3 recurrences of a specific Right-aptamer (designated as 288/10AptR1; see, FIG. 27) were found.

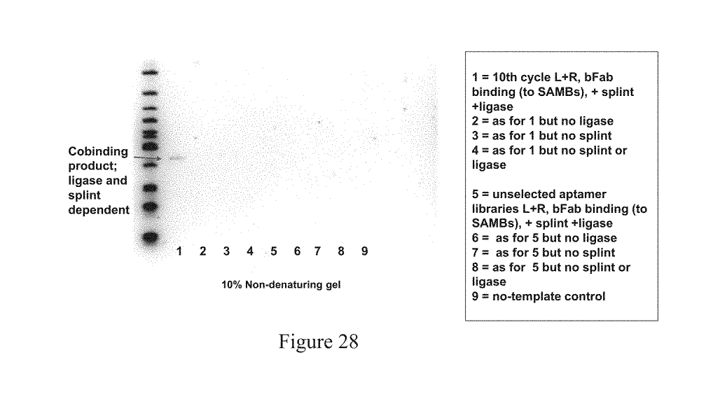

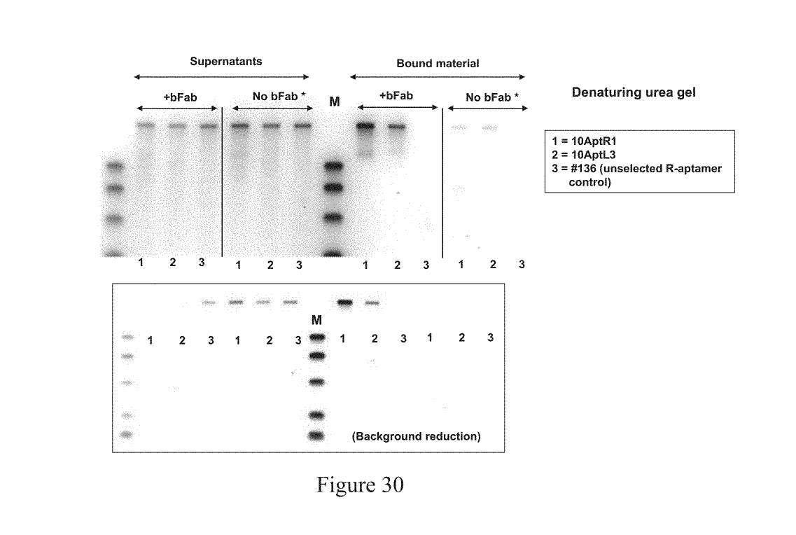

Left- and Right-10th cycle aptamer subpopulations from the Fab target were then subjected to the co-binding procedure on the Fab target (see, FIG. 12), as shown in FIG. 28. The amplified binary products were then sequenced and characterized. It was found that the Right-aptamer clone 228/0AptR1, previously observed as a recurrent singlet clone (see, FIG. 27), was also found independently in 5 independent binary clones. Notably, in one of these binary clones (10CB-10), the Left-aptamer component (229/10AptL3) had been previously independently isolated and sequenced from the 10th cycle Left-aptamer subpopulation (see, FIG. 30). The recurrence of identical sequences in both the singlet and binary aptamer subpopulations that had been selected for Fab binding was consistent with the expected reduction in the subpopulation size towards a set of aptamers with useful Fab-binding affinity.

In particular, FIG. 27 shows a 10th cycle analyses of aptamers binding biotinylated Fab. Recurring singlet aptamer clone 228 (10AptR1) is shown. The entire R-aptamer is shown with corresponding primer sequences (as indicated).

In particular, FIG. 28 shows a co-binding test of 10th cycle L- and R-aptamers to biotinylated Fab. A product was observed for the selected (10th cycle; Lane 1) aptamer pairs, but no such product were seen with primary (unselected) aptamers at this level of sensitivity. The cobinding process (shown schematically in FIG. 12) was equivalent to that used for 4th-cycle aptamers (see, FIG. 27).

In particular, FIG. 30 shows direct binding of specific 10th-cycle aptamers (10AprR1, 10AptL3) to bFab (Direct Binding Assay using streptavidin magnetic beads). Aptamers were incubated with bFab (2.5-fold molar excess), then bound to SAMBs. Supernatants were taken, and the SAMBs washed 3 times. Bound material was eluted with 0.1 M NaOH, and then precipitated, washed, dried, and reconstituted before loading samples on a denaturing acrylamide gel. “No biotinylated Fab” (bFab) indicates that no bFab was present during initial incubation, but aptamers were then treated with SAMBs in common with the (+) bFab tubes. M is a size marker (top band 60 bases; aptamer bands 85 bases).

### Example 3: Direct Demonstration of Binding of Specific 10th-Generation Singlet Aptamers Towards the Fab Target

To assess binding of 10^{th}-cycle singlet aptamers to the selective agent (the Fab target), direct binding assays were carried out. Here, single-stranded aptamer preparations (self-annealed as usual) were incubated with or without biotinylated Fab fragments in PBSM, followed by adsorption onto streptavidin magnetic beads. After this incubation period, the beads were magnetically separated, and the supernatants retained. After 3 washes of the beads, the bound material was eluted by means of 2×20 second incubations with 100 μl of 0.1 M NaOH, with the eluates immediately precipitated with 0.3 M sodium acetate and 3 volumes of ethanol. Pellets were washed with 70% ethanol, and dried. After reconstitution in 5 μl of TE, 1 μl samples were denatured in formamide and run on 10% urea (denaturing) gels. Results of such an experiment with the candidate singlet aptamers 228/10AptR1 (see, FIG. 27 and FIG. 29) and 229/10AptL3 (see, FIG. 29) and a specific arbitrary unselected control Right-aptamer (#136; sequence corresponds to: GCAAAGACATCTGGA CACGCCACTTATAGTCTACGTGAAGCACTGCGCTGGAACAGCCTAAAAAAGGAG AAGGAGACTTAGAGGC (SEQ ID NO:6); where underlining indicates the 40-mer aptamer tract; remaining sequences are primer sites) are shown in FIG. 30. The supernatants from the 228/10AptR1 and 229/10AptL3 binding (but not #136) were depleted only in the presence of biotinylated Fab. Moreover, the eluted material from biotinylated Fab on the streptavidin magnetic beads was highly enriched for aptamer bands only for 228/10AptR1 and 229/10AptL3 (see, FIG. 30). These results strongly suggest that the selected aptamers 228/10AptR1 and 229/10AptL3 showed significant and specific interaction with the Fab target.

In particular, FIG. 29 shows representative binary clone 10CB-01 obtained from 10th cycle co-binding experiment (see, FIG. 29). The Left-aptamer component of this binary (229/10AptL3, as shown) was previously independently isolated directly from the Left-singlet subpopulation; the Right-aptamer component (228/10AptR1) was previously independently isolated directly from the Left-singlet subpopulation (see, FIG. 28).

### Example 4: Direct Demonstration of Binding of Specific 10th-Generation Binary Aptamers Towards the Fab Target

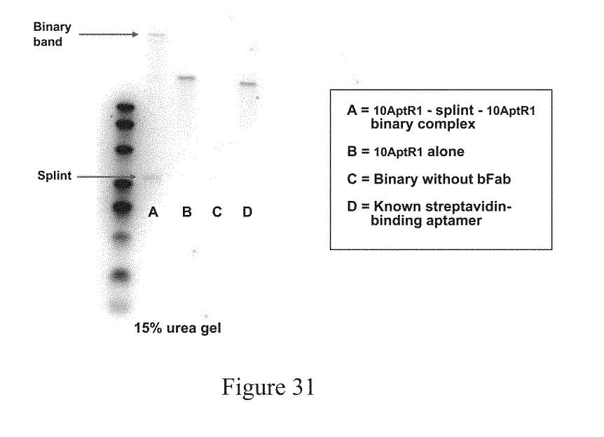

Despite successful in situ assembly of binary aptamers upon the target to which they were bound (as in Example 1), it could not be assumed that binary aptamers formed in solution would be capable of binding the same targets. This was assessed using the same aptamers as used in direct binding tests (Example 3; 228/10AptR1 and 229/10AptL3), but where the aptamers were initially ligated together by means of the same splint oligonucleotide as used in Example 1. Under the same experimental conditions as in Example 3, when equivalent samples were run on a denaturing gel, a bound band corresponding to the binary product was seen, as well as the corresponding splint oligonucleotide as expected (see, Lane A, FIG. 31). One of the component singlet aptamers (228/10AptR1) was used as a control, and a bound band was observed, as previously shown (see, Example 6, FIG. 30). No binding of the binary product to streptavidin beads alone was seen (see, Lane C, FIG. 31), indicating the requirement for Fab binding. In this case, an additional control was used with a known aptamer with binding affinity directly for streptavidin, and here a bound band was seen independently of the presence of Fab as predicted (see, Lane C, FIG. 31). In particular, FIG. 31 shows binding of biotinylated Fab target by binary form of known Fab-binding singlet aptamers.

### Example 5: Co-Binding Tests on IgG1 Antibody

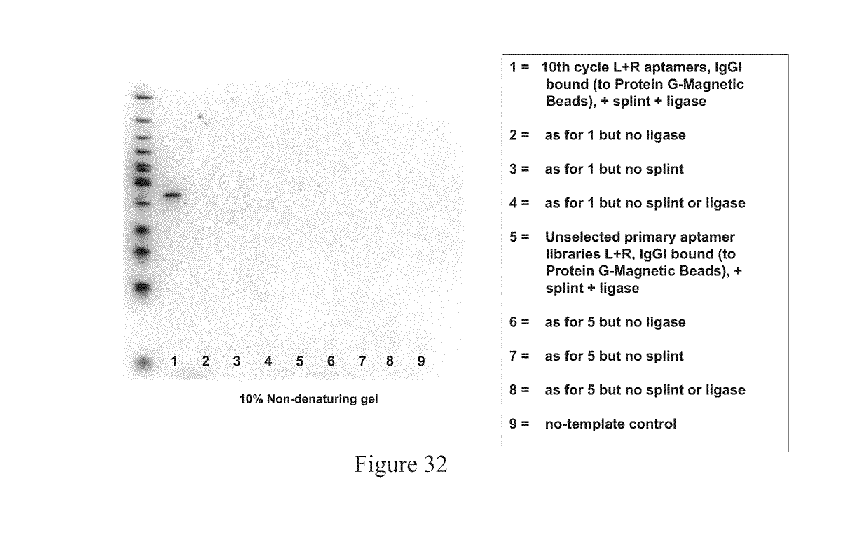

Although the target for selection in the above examples was biotinylated Fab, it was desirable that the derived aptamers from this process could also recognize intact immunoglobulin of the same isotype (murine gamma 1). This example serves as a generic demonstration of the use of a smaller component of a larger molecule or molecular complex to initially identify separate Left- and Right-binding aptamers, and then use these initial subpopulations to identify binary aptamers recognizing the larger desired target. Left- and Right-aptamer populations from the 10th cycle of selection on biotinylated Fab were used for co-binding testing on intact murine IgG1 (Santa Cruz Laboratories). This was carried out in an equivalent manner as for previous co-binding tests on biotinylated Fab (see, FIG. 27), but where the IgG1 replaced the Fab (in the same molar amounts), and the IgG1 itself was adsorbed to the sold phase by binding to Protein G magnetic beads (New England Biolabs). Following splint-mediated ligation, washing, and elution (as for Example 2), products were amplified (25 cycles) with primers defining ligated binary aptamers (see, FIG. 11), and analyzed on a non-denaturing acrylamide gel (see, FIG. 32). Results showed that the selected Left- and Right-aptamer populations gave rise to a binary product band, which was only produced when both the splint oligonucleotide and ligase were present (see, FIG. 32). No such easily detectable bands were observed from the primary (unselected) aptamer libraries.

In particular, FIG. 32 shows co-binding on IgG1 target of 10th-cycle Fab-selected aptamers. The method for co-binding (see, FIG. 12) was equivalent to that used in FIG. 27, except that IgG1 was the target rather than biotinylated Fab, and selection on solid-phase was accomplished by binding the IgG1 to Protein G-magnetic beads.

### Example 6: Effector Oligonucleotide Templating on Solid-Phase Templates

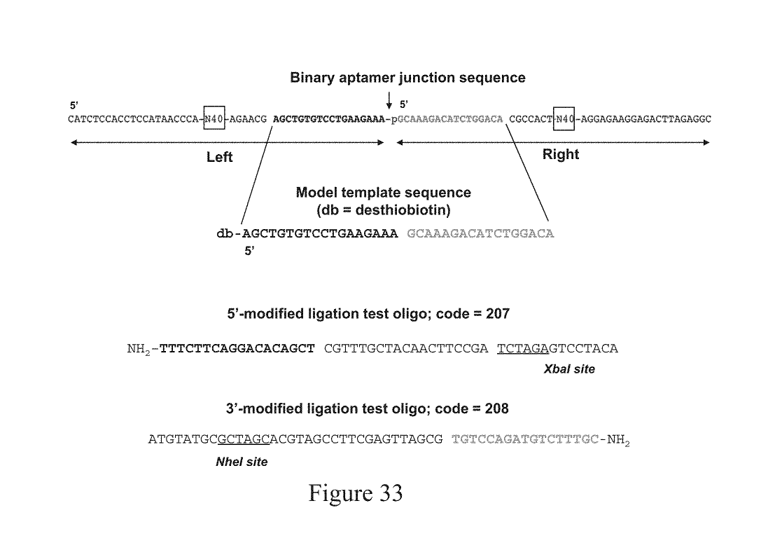

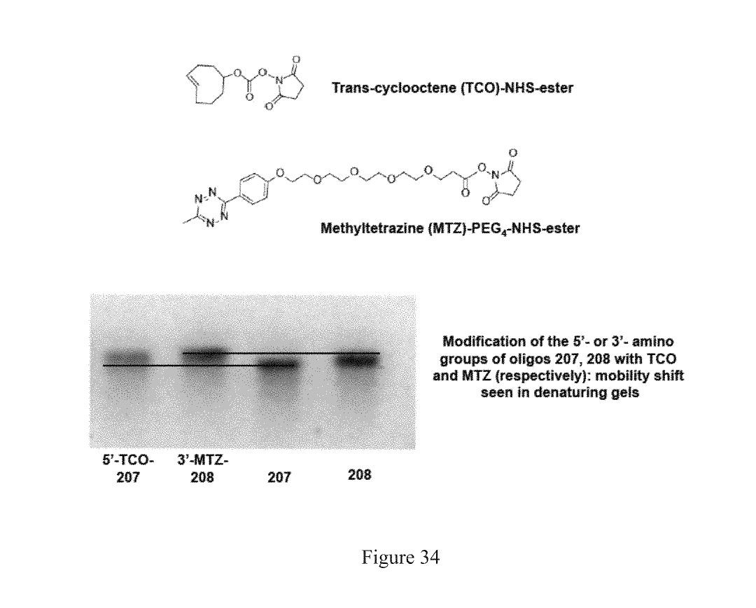

For aptamers to be useful for template assembly, they should display accessible sequences of sufficient length to act as templates for effector partials. The ability of the designated aptamer sequences to act as templates was initially assessed by means of corresponding desthiobiotinylated oligonucleotide sequences rendered solid-phase by capture on streptavidin magnetic beads. Sequences of the model template in relation to the aptamer junction region, and complementary test oligonucleotides, are shown in FIG. 33. As a convenient model for template assembly reactivity with traceless Staudinger chemistry, oligonucleotides modified with Inverse Electron-Demand Diels-Alder (IEDDA) chemical reactants were employed. In order to do this, oligonucleotides with 5′ or 3′ amino-modifications (see, FIG. 33) were reacted with N-hydroxysuccinimide-activated trans-cyclooctene (TCO) ester or corresponding methyltetrazine (MTZ) ester for 4 hours at room temperature in phosphate-buffered saline. After this, unreacted esters were removed by desalting columns (BioRad). The resulting oligonucleotide adducts could be distinguished from unreacted control corresponding oligonucleotides on denaturing gels via clear-cut mobility differences (see, FIG. 34).

In particular, FIG. 34 shows the structure of TCO and MTZ reagents for amino-terminal oligonucleotide derivatization, and demonstration of mobility shifts in modified 207 and 208 oligonucleotide products.

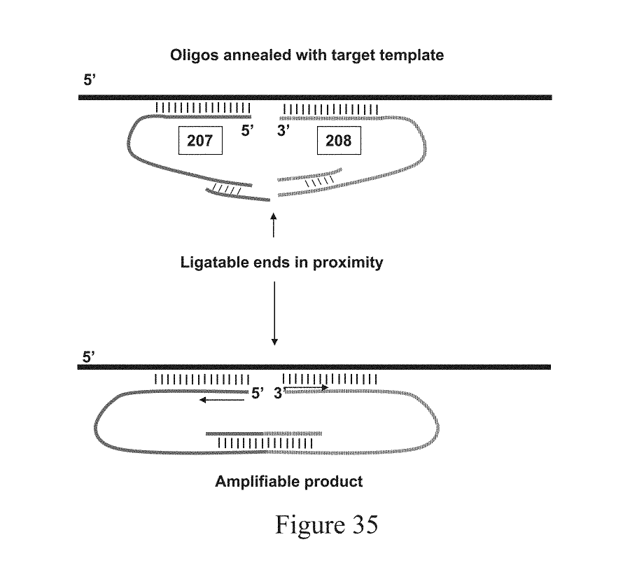

Although test oligonucleotides annealed to target templates and joined via IEDDA click chemistry cannot be directly amplified, the product can be amplified by inverse PCR if the opposite ends of the oligonucleotide pair are conventionally ligated. To effect this, the test template-complementary oligonucleotides (207 and 208, see, FIG. 33) were equipped with mutually compatible restriction sites. Prior to use in templating tests, the TCO-modified 207 and MTZ-modified 208 oligonucleotides (see, FIG. 33 and FIG. 34) were annealed with 28-mer oligonucleotides complementary to their 3′ and 5′ ends, respectively. (Oligonucleotide complementary to 3′ end of 207: TGTAGGACTCTAGATCGGAAGTT GTAGC (SEQ ID NO:7); Oligonucleotide complementary to 5′ end of 208: CTCGAAGGCTACGTGCTAGCGCATACAT (SEQ ID NO:8)). Following this, the partially-duplexed TCO-modified 207 and MTZ-modified 208 oligonucleotides were digested with Xba I and Nhe I, respectively. When these oligonucleotides mutually anneal to a template where their complementary sites are adjacent, the digested ends are in close proximity to each other and can be efficiently ligated by T4 ligase (see, FIG. 35).

In particular, FIG. 35 shows annealing of oligonucleotides 207 and 208 to target template, and resulting spatial proximity of duplexed ends with compatible restriction site overhangs. The ligated product is amplifiable by PCR, inverse with respect to the original 5′ and 3′ ends of the oligonucleotides.

TCO-modified 207 oligonucleotide bearing the above Xba I site 5′ overhang was annealed with desthiobiotinylated target (aptamer-junction) oligonucleotide, and then the material bound to streptavidin magnetic beads in phosphate-buffered saline with 1 mM MgCl₂ (PBSM). After three washes with PBSM, excess MTZ-modified 208 oligonucleotide was added and incubated 5 minutes at 37° C. and 1 hour at room temperature, followed by three more washes. Following this, the solid-phase magnetic bead preparations were washed twice in ×1 T4 DNA ligase buffer with 1 mM ATP (NEB), and split into two portions, with and without 400 units T4 DNA ligase. After 2 hours at room temperature, the preparations were washed in PBSM, and bound material was then eluted from the streptavidin magnetic beads by incubation with 100 μM D-biotin (Sigma). Samples were then run on a 10% denaturing acrylamide gel.

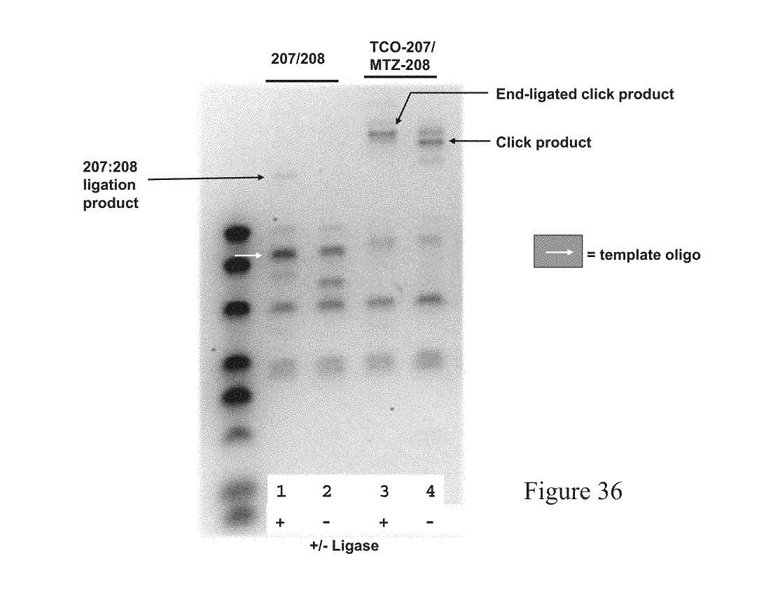

Results showed that IEDDA click product between the TCO-modified 207 and MTZ-modified 208 oligonucleotides formed on the solid-phase template (see, FIG. 36). This band was size-shifted by ligation of the restriction site ends (see, Lanes 3 vs. 4, FIG. 36), corresponding to an expected circularization process. Unmodified control oligonucleotides showed no band with the IEDDA product mobilities, but did show a ligation-specific band corresponding to restriction end joining (see, Lanes 1 vs. 2, FIG. 36).

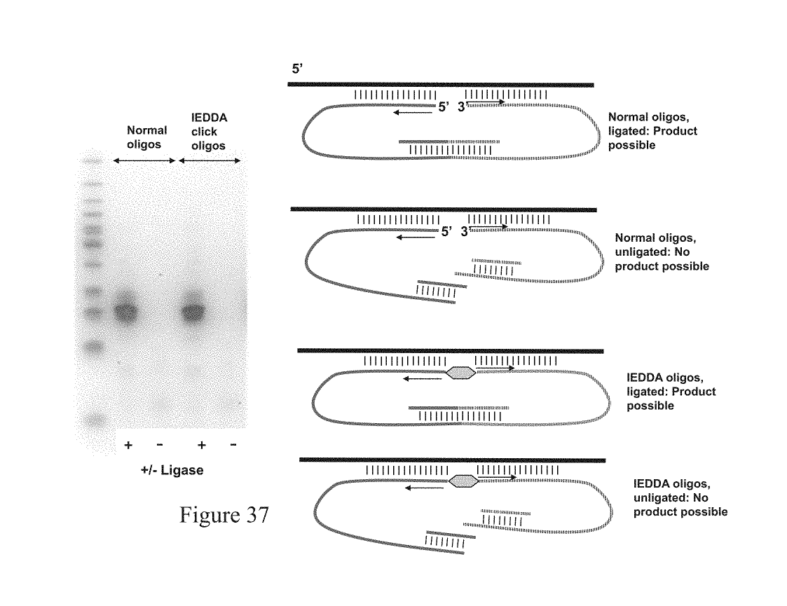

In particular, FIG. 36 shows solid-phase oligonucleotide-based templating using sequences present in aptamers. Template and oligonucleotide sequences are as for FIG. 34. It was subsequently shown with the same eluted material that PCR product formation (inverse with respect to the IEDDA joining site) was possible, but only after in situ ligation of the restriction ends as expected (see, FIG. 37). This demonstrated that inverse PCR is a suitable read-out for in situ templating of model templated assembly reactions.

In particular, FIG. 33 shows sequences of binary aptamer junctions and test aptamer template-directed ligation oligonucleotides. Boxed N40 sites are random regions of aptamers.

In particular, FIG. 37 shows PCR product formation for end-joined oligonucleotides in situ on solid-phase streptavidin magnetic beads (inverse with respect to IEDDA click join site).

### Example 7: Aptamer-Mediated Templating of Effector Oligonucleotides on Target

Following demonstration of templating on solid-phase oligonucleotides corresponding to the binary aptamer templating regions, it was shown that templating can be effected on aptamer templates themselves, while bound to specific targets in situ. Both L- and R-aptamers selected for binding biotinylated anti-BRD7 Fab (bFab) and arbitrary unselected control L- and R-aptamers were separately self-annealed and incubated in appropriate combinations (140 pmol of each aptamer; 25 μl final volumes) with and without 35 pmol of the bFab target (see, Table 1). After 1 hour at room temperature, the preparations were treated with 100 μl streptavidin magnetic beads for 30 minutes at room temperature with shaking (where beads were initially magnetically separated from the storage medium, washed twice with 1 ml of PBSM, and resuspended in the original volume of PBSM prior to use). Following this, beads were magnetically separated and washed once with 0.5 ml PBSM, and twice with 100 μl of ×1 SplintR® ligase buffer (New England Biolabs), and resuspended in 50 μl of SplintR® ligase buffer (with ATP) also containing 60 units murine ribonuclease inhibitor (NEB). Subsequently, 140 pmol (1.4 μl) of an RNA oligonucleotide was added, corresponding to the complement to the L/R inter-aptamer region (see, FIG. 11), with the sequence: UCCAGAUGUCUUUGCUUUCUUCAGGACACAG (SEQ ID NO:9). The preparations were annealed for 5 minutes at 37° C., and then at 30° C. for 1 hour, prior to the addition of 25 units of SplintR® ligase (New England Biolabs, a Chlorella ligase with high nick-sealing ability for DNA strands on RNA templates). After 1 hour at room temperature, the magnetic beads with the bound bFab/aptamer-RNA duplexes were washed once with 100 μl of RNase H buffer (New England Biolabs), and then resuspended in 50 μl of the same buffer with 5 units RNase H (New England Biolabs) for 10 minutes at 37° C. and 20 minutes room temperature. After washing once with 0.5 ml PBSM, samples were resuspended in 50 μl of PBSM. Then 105 pmol (5.3 μl; 3-fold molar excess over initial amount of bFab) of methyltetrazine-3′-modified oligonucleotide 208 (as in Example 6) for 30 minutes at room temperature, followed by washing with 0.5 ml PBSM and resuspension in 50 μl of PBSM. Subsequently, 105 pmol (5.3 μl; 3-fold molar excess over initial amount of bFab) of trans-cyclooctene-5′-modified oligonucleotide 207 (see, Example 6) were added, also for 30 minutes at room temperature. Preparations were then washed with 0.5 ml PBSM and bound DNA was eluted with two treatments with 100 μl of 0.1 M NaOH/5 mM EDTA for 20 seconds at room temperature (pooling of magnetically-separated supernatants), followed by immediate precipitation at −20° C. (for 30 minutes) with 0.3 M NaOAc, 20 μg glycogen, and 3 volumes of 100% ethanol. Preparations were washed with 1 ml of 70% ethanol, dried, and re-dissolved in 4.0 μl TE. Samples (1.0 μl) were run on 15% urea denaturing gels.

TABLE 1
Aptamer templating experiment (Example 7)

| | +/− L- | +/− R- | | | |
|---|---|---|---|---|---|
| | Aptamer | Aptamer | +/− bFab- | +/− RNA | +/− |
| Reaction no. | (code) | (code) | BRD7 | Splint | RNase H |
| 1 | +(229) | +(228) | + | + | + |
| 2 | +(229) | +(228) | + | − | − |
| 3 | +(229) | +(228) | − | + | + |
| 4 | +(229) | +(228) | + | + | + |
| 5 | +(229) | +(138) | + | + | + |
| 6 | +(139) | +(228) | + | + | + |
| 7 | − | − | + | + | + |
| 8 | − | − | − | + | + |

140 pmol of each aptamer was initially self-annealed, and then incubated in the reaction tubes, with or without 35 pmol biotinylated anti-BDR7 Fab (bFab; 40-fold aptamer excess). 229, 228: specific L- and R-bFab-binding aptamers; 139, 138: arbitrary L- and R-aptamer sequences not selected for bFab binding, shown in bold.

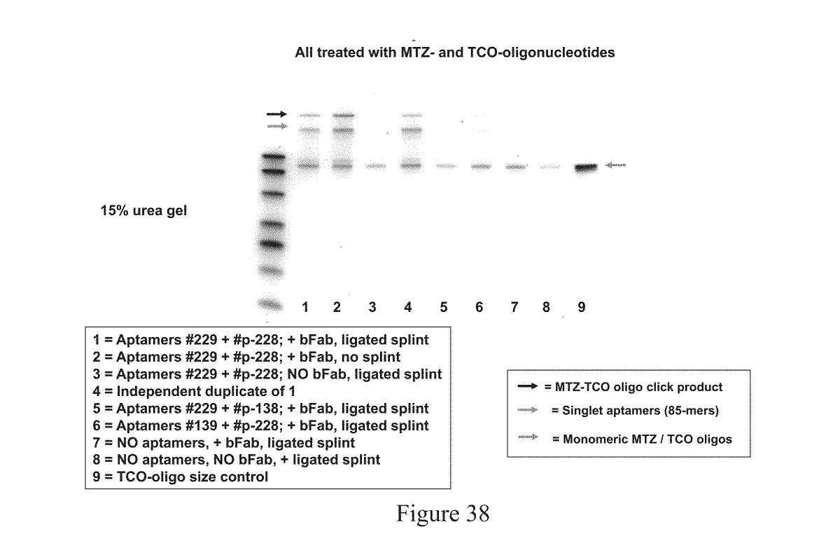

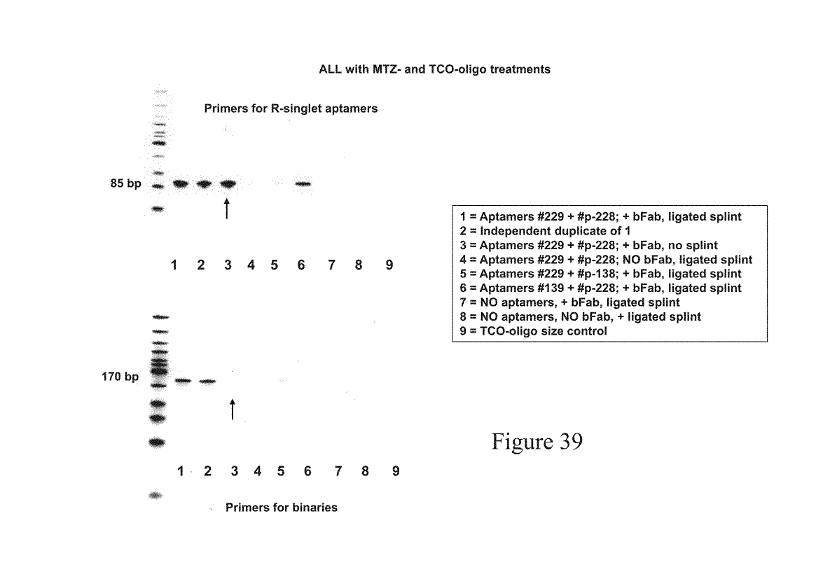

Gel analysis showed that reaction between the model click-labeled oligonucleotides was present on both specific aptamers as templates bound to bFab (see, FIG. 38, lanes 1, 2, and 4). However, in this case, splint-mediated ligation of L-(229) and R-(228) aptamers was unnecessary, as product was observed when splint/ligase was omitted (see, lane 2). That binary aptamers via splint-ligation were formed was shown with primers specific for both binary and (as an example of a singlet aptamer) R-aptamer forms (see, FIG. 11 for primer configurations). Singlet aptamers with the R-primers were only seen for preparations with the bFab-binding R-aptamer #228 as expected (see, FIG. 39). And binary products of #228 with its partner #229 were only observed when splint and ligase were applied (see, lanes 1 and 2, for the 170 bp binary band, FIG. 39).

In particular, FIG. 38 shows templating of model IEDDA click reactions by aptamer templates while bound to bFab target in situ. Aptamers #229 and #228 were originally selected as proximal binaries on bFab target (p-228 denotes the presence of a 5′ phosphate group to enable ligation with its partner aptamer via the RNA splint); Aptamers #139 and #138 are known non-binders.

In particular, FIG. 39 shows PCR testing of binding and binary formation of aptamers bound to bFab in situ. All preparations with #228 (known R-aptamer bFab binder; lanes 1, 2, 3 and 6) show good R-singlet bands. However, only the duplicate preps with splint+ligase showed the presence of the binary band. Lane 3 (unligated #228/#229) showed a strong R-singlet band (showing bFab binding) but no binary band (arrows).

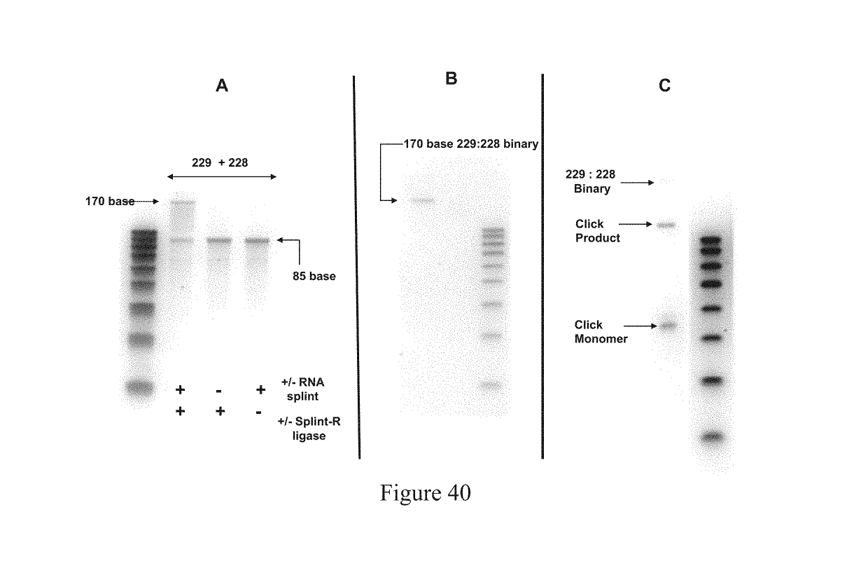

While aptamer-mediated proximity alone could promote the templated assembly of click-labeled oligonucleotides (see, FIG. 38), other templating applications may benefit from the contiguous longer template afforded by binary L-R aptamer pairs. Thus, pre-formed binary #229-#228 aptamer was prepared by annealing both aptamer strands with the above RNA template at high concentrations, and then removing the template with RNase H (see, FIG. 40, panel A). To remove remaining singlet strands, the 170-base binary strand was purified on an agarose gel (sample of purified material shown in FIG. 40, panel B). Subsequently, it was demonstrated that the assembled binary aptamer still bound to the target biotinylated Fab, and provided accessible template for the 207-208 labeled oligonucleotide click reaction (see, FIG. 40, panel C).

In particular, FIG. 40 shows the formation and testing of binary aptamers in situ on bFab target. Panel A shows RNA-splint-mediated formation of binary aptamers between singlet L- and R-aptamers #229 and #228 respectively, in solution at high concentration. Panel B shows purified sample (1.5% agarose) of 170 bp #229-#228 binary with the splint removed by RNase H (denaturing acrylamide gel), Panel C shows the formation of model click product on binary aptamers bound to specific bFab target. Addition of the click-labeled oligonucleotides 207 and 208 were as for FIG. 39.

### Example 8: Formation of Accessible Template in Binary Aptamers by Means of a Short Stem-Loop Bridge

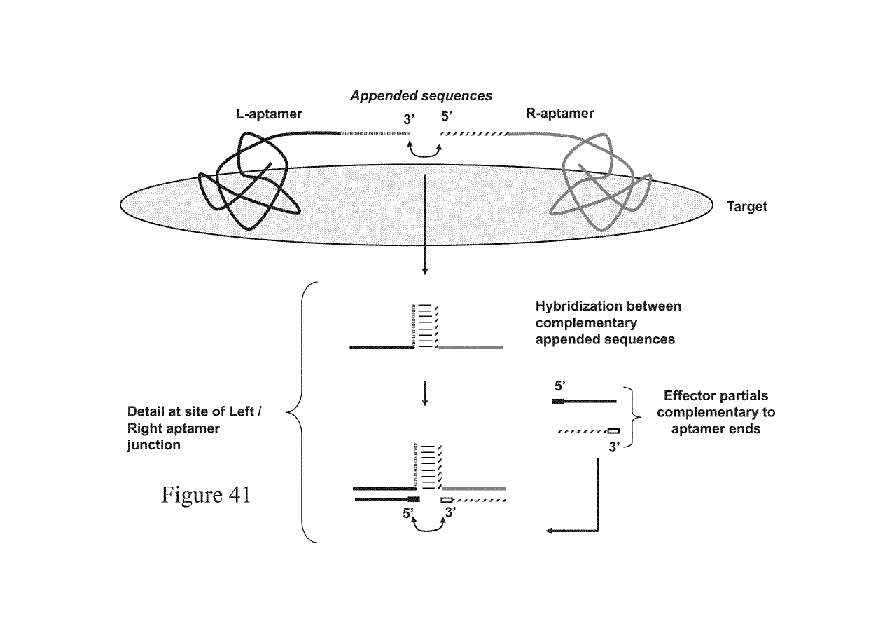

Although formation of binary aptamers can be effected in situ by means of a removable RNA splint (see, Example 6), an alternate method was developed where no ligation is necessary. Here, short complementary sequences were appended onto the 3′ and 5′ ends of L- and R-aptamers respectively, where they bind in proximity to a common target. As a consequence of this, the mutually complementary modified ends of the aptamers form a short stem-loop bridge, schematically depicted in FIG. 41. It is known that stem-loops can function for templating for template assembly purposes, demonstrated using model click oligonucleotides.

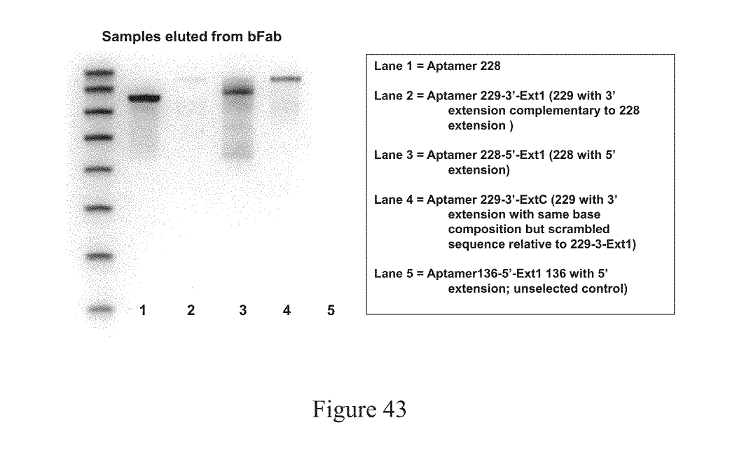

Alternately, binaries can be assembled via stem-loop hybridization in solution. The aptamer pair #229 (L) and #228 (R) targeting the biotinylated Fab-BRD7 protein were synthesized with mutually complementary 10-base 3′ and 5′ ends respectively (see, FIG. 42). Although the appended segment sequence is arbitrary, here a G/C sequence was used for maximum duplex stability. A short stem sequence is desirable to minimize the chance that the appended segment will interfere with aptamer function, and sequences complementary to the 40-base aptamer region are thus excluded. Nevertheless, the successful addition of an appended segment compatible with aptamer function should still be tested empirically. Shown in FIG. 43 is such a functional test for the appended aptamers. The #229 aptamer binding for the biotinylated Fab was reduced somewhat for the stem-loop tag, but less so for a control tag with the same base composition but scrambled sequence. The #228 aptamer was functionally little affected by the presence of the appended tag (see, FIG. 43).

In particular, FIG. 41 shows a schematic depiction of alternate in situ formation of template from a proximal binary aptamer pair by means of a short step-loop bridge. It is known that stem-loop structures in general can be used for model click oligonucleotide templated reactions.

In particular, FIG. 42 shows the structure of aptamers used for testing the complementary-end stem-loop bridge binary templating approach, and corresponding sequences. Primer sites are shown in black; aptamer 40-mer segments are shown in gray; and appended 10-mer sequences are shown in bold, also boxed.

In particular, FIG. 43 shows testing the effect of aptamer extensions on ability to bind bFab-BRD7. 140 pmol of self-annealed aptamers were incubated with 35 pmol bFab (25 μl final volume) at room temperature for 3.5 hours, then adsorbed onto 50 μl streptavidin magnetic beads in PBSM for 1 hour at room temperature. Supernatants were then magnetically removed, and the beads washed twice with 0.5 ml of PBSM. Bound material was eluted with 2×100 μl of 0.1 M NaOH/5 mM EDTA, precipitated with 20 μg of glycogen/0.3 M NaOAc, 3 volumes of ethanol, washed once with 1 ml of 70% ethanol, dried, and redissolved in 20 μl of TE. One μl samples were run directly on an 8 M urea denaturing gel.

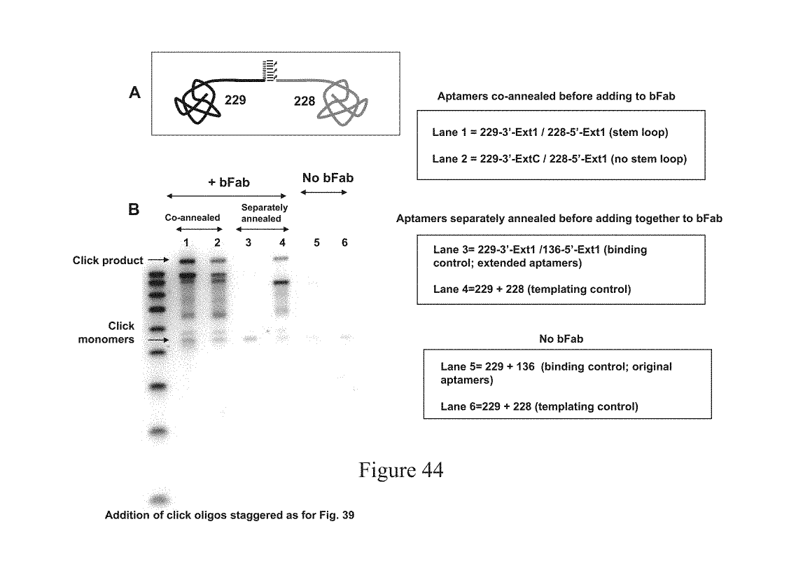

The extended aptamers were then assessed for their abilities to act as templates for model click reactions. Extended aptamers 229-3′-Ext1 and 228-5′-Ext1 were annealed together (3 minutes at 80° C., then 5 minutes at 0° C.), to allow aptamer self-annealing, and also inter-aptamer hybridization via the mutually complementary 10-base extensions (schematic depiction in FIG. 44, panel A). Control aptamers #229, #228, and 136-5′-Ext1 were separately self-annealed in the same manner. Aptamer preparations were incubated with bFab target (see FIG. 44, panel B), washed, and bond material eluted with sodium hydroxide as for FIG. 39. After precipitation, washing, and drying, eluted material was reconstituted in 10 μl TE, with 1 μl samples run on a denaturing urea gel. Results showed that proximal aptamers alone (spatially close but lacking a binary join) could enhance templated click reactivity (see, Lane 4, FIG. 44, panel B). Likewise, control aptamers with 3′ and 5′ extensions without mutual complementarity were capable of similar click activity promotion (see, Lane 2, FIG. 44, panel B). However, not only could the stem-loop linked binary preparations still support click activity (see, Lane 1, FIG. 44, panel B), but the product level was increased relative to the control lanes 2 and 4. Whether this is a consequence of improved target binding itself, or enhanced templating, the end result still indicates improved templating for template assembly.

In particular, FIG. 44 shows testing of the complementary-end binary stem-loop aptamer approach, with the extended aptamers 229-3′-Ext1 and 228-5′-Ext1. These aptamers were self-annealed and co-annealed simultaneously in solution to form the stem-loop linked binary (depicted schematically, panel A). Unextended aptamer controls were self-annealed separately as usual. Panel B shows results of templating tests with click-labeled oligonucleotides 207 and 208 (same protocol as for FIG. 34, FIG. 35, and FIG. 39).

The principles demonstrated within this example are analogous to, but distinct from, the L-DNA tagging procedure described above (see, FIG. 19).

### Example 9: Affinity Measurements for Specific Aptamers

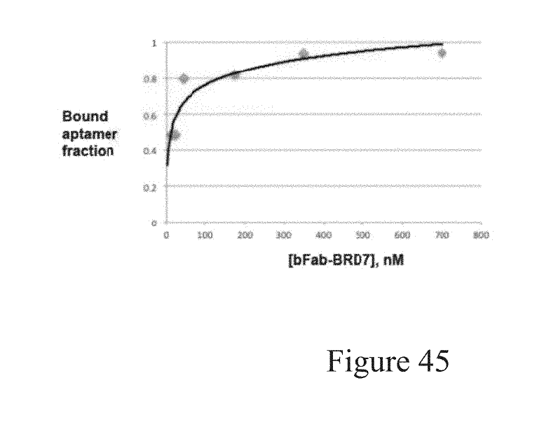

Aptamer affinities for defined targets are measurable by QPCR-based methods, in conjunction with a process for distinguishing bound from unbound aptamer over a range of target concentrations. This was applied to the aptamer #228, which was selected for bFab binding as a singlet, and binary binding with its partner #229. To construct a binding curve, dilutions of the bFab target (from 700 nM downwards in 2-fold dilutions) were incubated with a constant concentration of self-annealed #228 (10 nM) overnight (50 μl final volumes in PBSM) such that equilibrium conditions were attained. The preparations were then incubated with 75 μl of streptavidin magnetic beads (in molar excess over the highest concentration of bFab) for 1 hour at room temperature with shaking. Beads were then magnetically separated from supernatants, with each tube subjected to three washings with 0.5 ml of PBSM (original supernatants and washings were combined to give a total unbound fraction). Bound material was subsequently eluted from the beads by 2×20 second incubations with 0.1 M NaOH/5 mM EDTA, pooling the magnetically-separated eluate supernatants into a single tube. These were immediately precipitated with 20 μg glycogen/0.3 M NaOAc/3 volumes of ethanol (for 30 minutes at −20° C. incubation), followed by washing with 1.0 ml of 70% ethanol, drying, and reconstitution in 50 μl of PBSM. Samples of all preparations (1.0 μl) were then analyzed in triplicate in 96-well plates by QPCR, by means of a Bio-Rad CFX96 Touch instrument, with a cycle of 95° C. for 30 seconds; 40× (5 seconds at 95° C., and 30 seconds at 60° C.), in 20 μl volumes. Reaction mixes used ×1 BioRad iTaq PCR mixes and 6 pmol each of R-aptamer-specific primers. (Forward R-primer: GCAAAGACATCTGGACACGC (SEQ ID NO:10); Reverse R-primer: GCCTCTAAGTCTCCTTCTCCT (SEQ ID NO:11)). Wells were analyzed during cycling for real-time SYBR-green fluorescence, and CT values assigned. All runs included a standard curve of serial dilutions of #228 aptamer. Replicate results were averaged and bound and unbound CT values were derived for each data point, allowing calculation of total bound fractions. From a plot of these bound fraction values vs. corresponding [bFab], a non-linear regression curve could be derived (see, FIG. 45). In turn, a K_{d} estimate (of about 11 nM) could be obtained from the equation for the experimental curve (see, FIG. 45) where K_{d} corresponds to fraction bound=0.5 (Jing et al., Anal. Chim Acta, 2011, 686, 9-18).

In particular, FIG. 45 shows a binding curve for aptamer #228 and biotinylated Fab-BRD7. Non-linear regression curve equation is y=0.1179 ln(x)+0.2181.

### Example 10: Aptamer-Mediated Surface Assembly of a T Cell HLA-A2 Restricted Epitope

Aptamers can be used to adapt a wide variety of surface structures as templates for the template assembly process, and this adaptation process can include multiple recognition molecules, in a “sandwich” type of arrangement. This example discloses the use of a biotinylated target molecule towards which a binary aptamer pair is directed. It also uses a biotinylated primary recognition molecule binding to a desired and pre-defined cell-surface marker, and a multivalent biotin-binding bridging molecule.

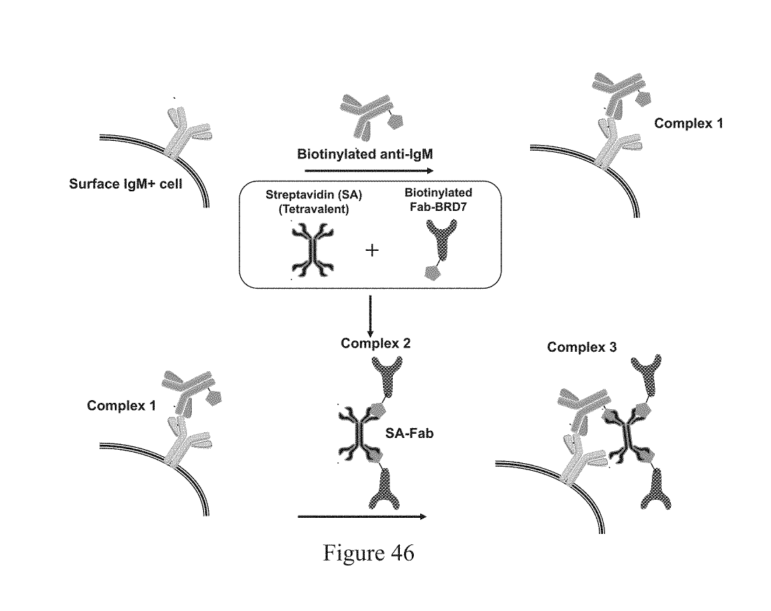

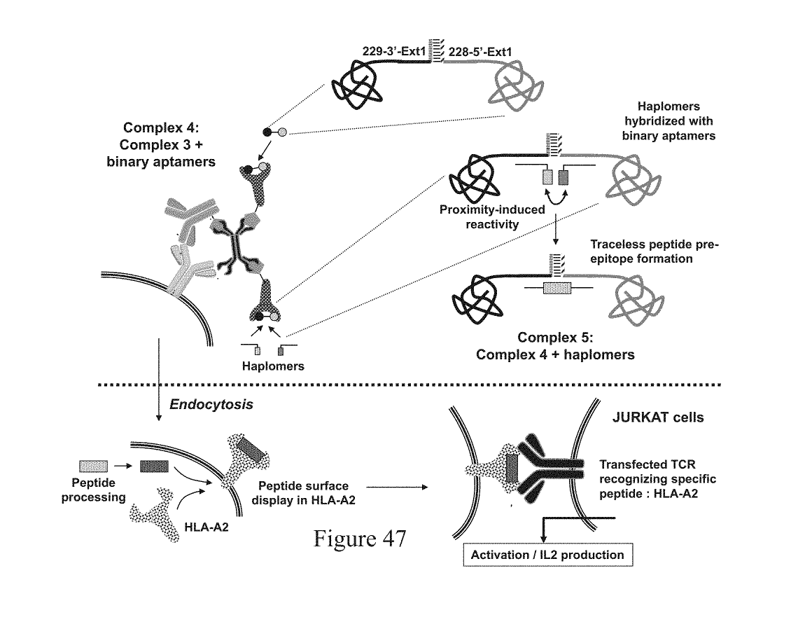

In this instance, the target molecule is a biotinylated anti-BRD7 Fab (see, Examples 1-4 and 7-9), the primary recognition molecule is a biotinylated anti-IgM (BD-Pharmingen), and the bridging molecule is a streptavidin-phycoerythrin conjugate (SA-PE; Fitzgerald Industries). Streptavidin alone (Sigma-Aldrich) can also be used in lieu of the phycoerythrin conjugate. Cells of interest (10⁶) expressing surface IgM (EBV-transformed lymphoblastoid cell lines) are harvested, washed with ×1 PBS, and treated for 1 hour at room temperature with biotinylated anti-IgM at a suitable concentration (as recommended by the manufacturer). Following 3×PBS washes, 100 pmol of SA-PE previously complexed in an appropriate molar ratio with biotinylated anti-BRD7 Fab (bFab) is incubated with the primed cell suspension for 1 hour at room temperature, with occasional resuspension of the cells. The pre-assembled SA-PE complex is created in the following manner: 50 pmol SA-PE is incubated with 50-100 pmol bFab for 1 hour at room temperature, in 1×PBSM. Since SA is tetravalent, this ensures that all bFab is bound without saturating the available SA biotin-binding sites. These steps are depicted in FIG. 46. The cells are then washed twice with PBSM, and resuspended in 0.5 ml of PBSM. Pre-annealed aptamers 229-3′-Ext1 and 228-5′-Ext (forming a binary via a stem-loop bridge, as in Example 8; 100 pmol) are added to the primed cells for 1 hour at room temperature, and washed twice with 1.0 ml of PBSM. The resulting complex is depicted in FIG. 47.

The success of the formation of the multi-layered sandwich is assayed at two levels. The presence of the target surface antigen (IgM) is demonstrated by subjecting complexed cell samples (primary anti-IgM antibody/SA-PE/bFab/binary aptamer) to flow analysis for fluorescence in the PE channel, in comparison with control cells treated in an identical manner except for the exclusion of the primary anti-IgM antibody. Aptamer binding is demonstrated with a bilabeled fluorescent splint oligonucleotide (as for the DNA splint in Example 1 (DNA splint (5′-TCCAGATGTCTTTGCTTTCTTCAGGACACAG (SEQ ID NO:12)) except for its modification at both 5′ and 3′ ends with fluorescent FAM moieties). The fluorescent splint (100 pmol) is incubated in PBSM with fully complexed cells and controls identical except for exclusion of the bFab for 1 hour at room temperature, and then cells are washed three times with 0.5 ml of cold PBSM before being subjected to flow analysis with the fluorescein channel.

Preparations passing these tests can be used for assembly of a Melan A/MART epitope (ELAGIGILTV (SEQ ID NO:13)) presented by HLA-A2, since the binary aptamer templating regions in this system (see, FIG. 11) are designed to hybridize with the haplomer Human-Papillomavirus-derived sequences described in the application PCT International Publication WO 14/197547.

Complexes on EBV-transformed HLA-A2+ cells expressing surface IgM recognized by the primary biotinylated antibody are equipped with the binary stem-loop aptamers as detailed above in this Example. Following washing as above, preparations are incubated with both haplomers recognizing the binary aptamer templating region for 1 hour at room temperature, and bearing MART epitope half-peptides. During this incubation, the haplomers hybridize to the aptamer surface template in proximity to each other, allowing formation of intact assembled epitope peptide. Cells are then washed with 1 ml of PBSM, and incubated for a further 2 hours at room temperature to allow endocytosis to occur (see, FIG. 47). Following this, treated cells are used to gauge uptake, processing, and HLA-A2 presentation of assembled peptides. This is performed using Jurkat cells transfected with a T cell receptor recognizing ELAGIGILTV in the context of HLA-A2, where the read-out for Jurkat activation is the secretion of IL-2, as described by Haggerty et al., Assay Drug Dev. Technol., 2012, 10, 187-201.

The process illustrated by this Example can encompass numerous other embodiments, involving variations on aptamers and targeting, and the types of structures assembled as the products of pairs of haplomers. Thus, aptamers may target cell surface structures directly, or any other components of a succeeding sandwich structure (as in FIGS. 46 and 47). Alternate haplomer-assembled products can included peptides binding to any other MHC class, or structures designed for direct recognition by antibodies. In the latter class of embodiments, such haplomer-assembled compounds include natural peptides, peptidomimetic structures, or non-peptide small organic molecules. Antibodies targeting such aptamer-mediated structures assembled from haplomers via the template assembly process can in turn promote target cell killing in various ways, including, but not limited to, antibody-dependent cellular cytotoxicity, complement pathways, or via antibody conjugates with highly cytotoxic drugs, including, but limited to, calicheamycin A and emtansine.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A singlet nucleic acid aptamer comprising:
a first portion folded into a tertiary structure that is able to bind to a target molecule; and
a second portion comprising either the 3′ or 5′ terminal region, wherein the second portion is hybridized to a first haplomer and a second haplomer;
wherein the first haplomer comprises:
a hybridization region that is hybridized to the second portion of the singlet nucleic acid aptamer; and
a reactive effector moiety;
wherein the second haplomer comprises:
a hybridization region that is hybridized to the second portion of the singlet nucleic acid aptamer; and
a reactive effector moiety;
wherein the reactive effector moiety of the first haplomer is in spatial proximity to the reactive effector moiety of the second haplomer.

2. The singlet nucleic acid aptamer of claim 1, wherein both the first portion and second portion comprise a primer binding site at their terminal ends.

3. A dual proximal nucleic acid aptamer pair comprising:
a first nucleic acid aptamer comprising:
a first portion folded into a tertiary structure that is able to bind to a target molecule; and
a second portion comprising the 3′ terminal region, wherein the second portion is hybridized to a first haplomer, wherein the first haplomer comprises:
a hybridization region that is hybridized to the second portion of the first nucleic acid aptamer; and
a reactive effector moiety; and
a second nucleic acid aptamer comprising:
a first portion folded into a tertiary structure that is able to bind to a target molecule; and
a second portion comprising the 5′ terminal region, wherein the second portion is hybridized to a second haplomer, wherein the second haplomer comprises:
a hybridization region that is hybridized to the second portion of the second nucleic acid aptamer; and
a reactive effector moiety;
wherein the reactive effector moiety of the first haplomer is capable of interacting with the reactive effector moiety of the second haplomer.

4. The singlet nucleic acid aptamer of claim 1, wherein both the first portion and second portion of each aptamer comprise a primer binding site at their terminal ends.

5. The dual proximal nucleic acid aptamer pair of claim 3 wherein the aptamer pair bind to the same target molecule such that the aptamer pair is in physical proximity.

6. The dual proximal nucleic acid aptamer pair of claim 3 wherein the aptamer pair bind to the different target molecules on the same cell such that the aptamer pair is in physical proximity.

7. The dual proximal nucleic acid aptamer pair of claim 3 wherein the aptamer pair bind to the different target molecules on different cells such that the aptamer pair is in physical proximity.

8. The dual proximal nucleic acid aptamer pair of claim 3 wherein the 5′ and 3′ terminal ends of the aptamer pair are ligated together.

9. A binary nucleic acid aptamer comprising:
a first portion folded into a tertiary structure that is able to bind to a target molecule;
a second portion folded into a tertiary structure that is able to bind to a target molecule;
a third portion located between the first and second portion, wherein the third portion is hybridized to a first haplomer and a second haplomer;
wherein the first haplomer comprises:
a hybridization region that is hybridized to the third portion of the binary nucleic acid aptamer; and
a reactive effector moiety;
wherein the second haplomer comprises:
a hybridization region that is hybridized to the third portion of the binary nucleic acid aptamer; and
a reactive effector moiety;
wherein the reactive effector moiety of the first haplomer is in spatial proximity to the reactive effector moiety of the second haplomer.

10. The binary nucleic acid aptamer of claim 9 wherein the first portion folded into a tertiary structure that is able to bind to a target molecule and the second portion folded into a tertiary structure that is able to bind to a target molecule each comprise about 20 nucleotides to about 80 nucleotides in length and have a Tₘ from about 55° to about 65° C., and the third portion located between the first and second portion comprises from about 40 nucleotides to about 60 nucleotides in length.

11. The nucleic acid aptamer of claim 1 wherein the nucleic acid comprises DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, or other nucleic acid analogues capable of base-pair formation, or any combination thereof.

12. The nucleic acid aptamer of claim 1 wherein the hybridization region of the haplomer and the portion of the aptamer which hybridizes to the hybridization region of the haplomer both comprise L-DNA.

13. The nucleic acid aptamer of claim 1 wherein the target molecule is intracellular.

14. The nucleic acid aptamer of claim 1 wherein the target molecule is on a cell surface.

15. The nucleic acid aptamer of claim 1 wherein the hybridization region of the first haplomer and/or the first haplomer comprises from about 10 to about 18 nucleotides in length.

16. The nucleic acid aptamer of claim 1 wherein the first haplomer and the second haplomer are covalently joined to their respective 3′ and 5′ ends.

17. A population of nucleic acid aptamers comprising two or more of the nucleic acid aptamers of claim 1 wherein the 5′ or 3′ ends of the aptamers are selected for accessibility to pairs of first and second haplomers.

18. An aptamer comprising the nucleotide sequence set forth in SEQ ID NO:1, SEQ ID NO:2, SEQ ID NO:3, or SEQ ID NO:4.

19. A method of selecting an aptamer from a library comprising:
binding of members of the library to a desired solid phase target;
washing the solid phase target;
eluting the bound members of the library;
precipitating the bound members of the library;
reconstituting the bound members of the library;
testing the bound members of the library for the best amplifiable region;
performing preparative asymmetric PCR;
testing the PCR products on a gel;
binding the PCR products to streptavidin magnetic beads;
washing the streptavidin magnetic beads;
eluting the top strands;
testing the eluted strands on a gel; and
performing the cycle nine to ten times until diversity of the binding aptamer population is sufficiently reduced such that analysis of the binding properties of specific predominant aptamer clones can be performed.

20. The method of claim 19 wherein the preparative asymmetric PCR comprises:
amplifying the selected aptamer population where the bottom strand corresponding to the aptamer complement is biotinylated; and
performing asymmetric PCR for differential amplification of the top strands, whereby a large molar excess of the top-strand primer is used, resulting in generation of an excess of single strands corresponding to the desired aptamer subpopulation.

21. The method of claim 20 wherein any biotinylated strands are removed by binding to solid-phase streptavidin, with the unbound supernatants containing the appropriate single-stranded preparation.

22. A method of selecting an aptamer having an accessible 3′ or 5′ terminal end for hybridization to a haplomer comprising:
contacting an aptamer with a corresponding target molecule;
contacting the aptamer with an oligonucleotide probe having a region that is complementary to the 3′ or 5′ terminal end of the aptamer, wherein the oligonucleotide probe is conjugated to biotin;
washing the aptamer-oligonucleotide probe complex to remove unbound oligonucleotide probe;
contacting the aptamer-oligonucleotide probe complex with streptavidin magnetic beads; and
washing the streptavidin magnetic beads and eluting the aptamer, wherein the aptamer possesses an accessible 3′ or 5′ terminal end for hybridization to a haplomer.

23. A method of preparing a binary aptamer comprising:
contacting a target molecule or target cell with a plurality of aptamers;
eluting the bound aptamers, including at least one left aptamer and at least one right aptamer;
contacting the target molecule or target cell with the population of bound left and right aptamers;
contacting the bound aptamers with a ligase and an RNA splint; and
removing the splint with RNase H; thereby resulting in a covalently ligated binary aptamer.

24. The method of claim 23 wherein the ligase is T4 DNA ligase or Chlorella DNA ligase.

25. The method of claim 19 wherein the aptamers are selected to bind to a cancer cell, and wherein aptamers that bind to normal cells are subtracted.

26. A method of delivering at least one aptamer to a pathogenic cell, said method comprising administering a therapeutically effective amount of any one or more aptamers of claim 1 to the pathogenic cell, wherein at least one active effector structure in the pathogenic cell is produced.

27. The method of claim 26, wherein the aptamer is administered separately from the haplomers.

28. The method of claim 26 wherein at least one of programmed cell death of the pathogenic cell, apoptosis of the pathogenic cell, non-specific or programmed necrosis of the pathogenic cell, lysis of the pathogenic cell, and growth inhibition of the pathogenic cell is produced.

29. The method of claim 26 wherein the pathogenic cell is selected from the group consisting of a virus infected cell, a tumor cell, a cell infected with a microbe, and a cell that produces a disease-inducing or disease modulating molecule that may cause inflammation, allergy or autoimmune pathology.

30. The method of claim 29 wherein the pathogenic cell is a virus infected cell and the method produces at least one of programmed cell death of the virus infected cell, apoptosis of the virus infected cell, non-specific or programmed necrosis of the virus infected cell, lysis of the virus infected cell, inhibition of viral infection, and inhibition of viral replication.

31. The method of claim 29 wherein the pathogenic cell is a tumor cell and the method produces at least one of programmed cell death of the tumor cell, apoptosis of the tumor cell, non-specific or programmed necrosis of the tumor cell, lysis of the tumor cell, inhibition of the tumor cell growth, inhibition of oncogene expression in the tumor cell, and modification of gene expression in the tumor cell.

32. The method of claim 29 wherein the pathogenic cell is a microbe-infected cell and the method produces at least one of programmed cell death of the microbe-infected cell, apoptosis of the microbe-infected cell, non-specific or programmed necrosis of the microbe-infected cell, lysis of the microbe-infected cell, inhibition of microbial infection, and inhibition of microbe replication.


SEQUENCE LISTING (13 sequences)

SEQ ID NO: 1 - DNA, 85 bp, Artificial Sequence
  Aptamer 229 (10AptL3)
catctccacctccataacccacggacgggcgtctagagaagtaggctgaaatatcgtggcgagaacgagctgtgtcctgaagaaa

SEQ ID NO: 2 - DNA, 85 bp, Artificial Sequence
  Aptamer 228 (10AptR1)
gcaaagacatctggacacgccactaagtggaggtgatctgtacttcatttatgagatcgcggcgaggagaaggagacttagaggc

SEQ ID NO: 3 - DNA, 95 bp, Artificial Sequence
  Aptamer 229-3′-Ext1
catctccacctccataacccacggacgggcgtctagagaagtaggctgaaatatcgtggcgagaacgagctgtgtcctgaagaaaccggctgcgc

SEQ ID NO: 4 - DNA, 95 bp, Artificial Sequence
  Aptamer 228-5′-Ext1
cgacgcgggcgcaaagacatctggacacgccactaagtggaggtgatctgtacttcatttatgagatcgcggcgaggagaaggagacttagaggc

SEQ ID NO: 5 - DNA, 31 bp, Artificial Sequence
  DNA splint
tccagatgtctttgctttcttcaggacacag

SEQ ID NO: 6 - DNA, 85 bp, Artificial Sequence
  Arbitrary control Right-aptamer
gcaaagacatctggacacgccacttatagtctacgtgaagcactgcgctggaacagcctaaaaaaggagaaggagacttagaggc

SEQ ID NO: 7 - DNA, 28 bp, Artificial Sequence
  Oligo complementary to 3′ end of TCO-modified 207
tgtaggactctagatcggaagttgtagc

SEQ ID NO: 8 - DNA, 28 bp, Artificial Sequence
  Oligo complementary to 5′ end of MTZ-modified 208
ctcgaaggctacgtgctagcgcatacat

SEQ ID NO: 9 - RNA, 31 nt, Artificial Sequence
  oligo complement to the L/R inter-aptamer region
uccagaugucuuugcuuucuucaggacacag

SEQ ID NO: 10 - DNA, 20 bp, Artificial Sequence
  Forward R aptamer primer
gcaaagacatctggacacgc

SEQ ID NO: 11 - DNA, 21 bp, Artificial Sequence
  Reverse R aptamer primer
gcctctaagtctccttctcct

SEQ ID NO: 12 - DNA, 31 bp, Artificial Sequence; identical to SEQ ID NO: 5
  Splint oligonucleotide

SEQ ID NO: 13 - protein, 10 aa, Artificial Sequence
  Melan A/MART epitope
ELAGIGILTV